(19) United States
(12) Patent Application Publication
(10) Pub. No.: US 2022/0290237 A1
(43) Pub. Date: Sep. 15, 2022

(54) METHOD FOR PREDICTING THE RESPONSE OF ANTIPSYCHOTIC DRUGS

(71) Applicants: INSERM (INSTITUT NATIONAL DE LA SANTÉ ET DE LA RECHERCHE MÉDICALE), Paris (FR); UNIVERSITÉ PARIS-EST CRÉTEIL VAL DE MARNE, Creteil (FR)

(72) Inventors: Stéphane JAMAIN, Creteil (FR); Réjane TROUDET, Paris (FR)

(21) Appl. No.: 17/606,099

(22) PCT Filed: Apr. 23, 2020
(86) PCT No.: PCT/EP2020/061314

§ 371 Date: Oct. 25, 2021

(30) Foreign Application Priority Data
Apr. 24, 2019 (EP) 19305528.2

(51) Int. Cl. C12Q 1/6883; A61K 31/5513

(52) U.S. Cl. CPC C12Q 1/6883; A61K 31/5513; C12Q 2600/106; C12Q 2600/156; C12Q 2600/158

(57) ABSTRACT

A fundamental shortcoming in the current treatment of schizophrenia is the lack of valid criteria to predict who will respond to antipsychotic treatment. The identification of blood-based biological markers of the therapeutic response would enable clinicians to identify the subgroup of patients in whom conventional antipsychotic treatment is ineffective and offer alternative treatments. As part of the Optimization of Treatment and Management of Schizophrenia in Europe (OPTiMiSE) programme, the inventors conducted a transcriptome analysis on 188 subjects with first episode psychosis, all of whom were subsequently treated with amisulpride for 4 weeks. They identify 32 genes for which the expression changed after treatment in good responders only. Among these genes, the expression of ALPL, a gene involved in vitamin B6 metabolism, as well as CA4, DGTA2, DHRS13, HOMER3 and WLS showed a significant difference in expression level between good and poor responders before starting treatment, allowing to predict treatment outcome with a predictive value of 93.8% when combined with clinical features Collectively, these findings identified new mechanisms to explain symptom improvement after amisulpride medication and highlight the potential of combining gene-expression profiling with clinical data to predict treatment response in first episode psychoses.

### FIELD OF THE INVENTION

The present invention relates to a method for predicting the response of antipsychotic drugs in patient in need thereof.

### BACKGROUND OF THE INVENTION

Since their introduction in the 1950s, antipsychotic drugs are the medication of choice in the treatment of psychoses. However, despite the subsequent introduction of many new antipsychotics, about one third of patients are relatively unresponsive to treatment (1-3). Inter-individual differences in clinical outcome following antipsychotic medication may depend on several factors (4), including doctor-patient relationship, pathogenic mechanisms, pharmacological factors (dosage, interactions, metabolism), clinical heterogeneity (diagnosis, age of onset), demographic descriptors (age, sex, ethnic origin, social status), environmental risk factors (traumatic events, drug abuse), inflammation background (5) and genetic factors (6). However, the basis of the heterogeneous response to treatment remains unclear. The lack of treatment algorithms or biomarker-based guidelines results in a trial-and-error process in order to find, for each patient, the adequate treatment at the optimal dose with a minimum of side-effects. As a result, determining whether a patient will respond to antipsychotics involves the careful evaluation of at least 2 courses of different treatments, which substantially delays the provision of alternative treatments such as clozapine (7).

Over the last decades, considerable research efforts have focused on whether genetic information could be helpful to predict patient's response as well as adverse effects to a given antipsychotic. Pharmacogenetic studies have mainly focused on specific candidate genes involved in the pharmacodynamics and in the pharmacokinetics of antipsychotic drugs or on the primary targets of antipsychotics (8). Although many genetic variants have been reported as associated with response to treatment, none of these studies have led to the identification of a biomarker robust enough to be applicable in clinical practice (9-15). More recently, genome-wide association studies (GWAS) of antipsychotic treatment response have used clinical scales or neurocognitive tests as outcome measurements, but without consistent findings and no functional validation of associated variants (16-24). Transcriptome analysis provides an alternative method of identifying biomarkers of treatment response, but to date, studies that used this approach had a limited statistical power (a maximum average power of 0.65 to detect fold change higher than 1.3) (25-27).

### SUMMARY OF THE INVENTION

A fundamental shortcoming in the current treatment of schizophrenia is the lack of valid criteria to predict who will respond to antipsychotic treatment. The identification of blood-based biological markers of the therapeutic response would enable clinicians to identify the subgroup of patients in whom conventional antipsychotic treatment is ineffective and offer alternative treatments. As part of the Optimization of Treatment and Management of Schizophrenia in Europe (OPTiMiSE) programme, the inventors conducted a transcriptome analysis on 188 subjects with first episode psychosis, all of whom were subsequently treated with amisulpride for 4 weeks. Total RNA was analysed by RNA-seq in each patient before and after treatment, and patients were genotyped using high throughput genotyping DNA chips.

Herein, the inventors identified 32 genes for which the expression changed after treatment in good responders only. These findings were replicated in an independent sample of 24 patients with first episode psychosis. Six genes (ALPL, CA4, DHRS13, HOMER3, CA4, DGAT2 and WLS) showed a significant difference in expression level between good and poor responders before starting treatment, allowing to predict treatment outcome with a predictive value of 93.8% when combined with clinical features. The inventors recently shown that change from amisulpride to olanzapine did not improve outcome for most of the patient suggesting that these gene expression level-based predictions may help in selecting patients to treat earlier with clozapine.

Collectively, these findings identified new mechanisms to explain symptom improvement after amisulpride medication and highlight the potential of combining gene-expression profiling with clinical data to predict treatment response in first episode psychoses.

### DETAILED DESCRIPTION OF THE INVENTION

A first aspect of the invention relates to a method for predicting antipsychotic treatment response of a patient in need thereof, comprising: i) determining, in a sample obtained from the patient, the expression level of at least one genes selected in the group consisting in AC073172.1, AC092171.4, AC132872.1, ACSL5, AL133351.4, AL391832.3, ALG1L13P, ALPL, AP000640.1, C15orf54, CA4, CXCR6, CYSLTR2, DGAT2, DHRS13, FAT1, FBXL13,GALNT14, GUCY1B3, HOMER3, KAZN, KIAA0319, LINC00963, NFE4, NLRP12, NLRP6, P2RY12, P4HA2, PLB1, SLC4A4, TRPC6, and WLS; ii) comparing the expression of the genes determined at step i) with a reference values and iii) concluding that the patient will not respond to antipsychotic treatment when the expression level determined at step i) is significantly different from the reference value.

Indeed, the inventors identified 32 genes for which the expression changed after treatment in good responders only. They showed that the genes C15orf54, TRPC6, CXCR6, CYSLTR2, FAT1, P2RY12, SLC4A4, ACSL5 and GUCY1B3 are up-regulated after 4-week treatment with amisulpride in responders. They also showed that the expression level of AC073172.1, AC092171.4, AC132872.1, AL133351.4, AL391832.3, ALG1L13P, ALPL, AP000640.1, CA4, DGAT2, DHRS13, FBXL13, GALNT14, HOMER3, KAZN, KIAA0319, LINC00963, NFE4, NLRP12, NLRP6, P4HA2, PLB1 and WLS are down-regulated after 4-week treatment with amisulpride in responders.

According to the invention, it will be concluded than the patient will not respond to antipsychotic treatment when the expression level of at least one genes selected in C15orf54, TRPC6, CXCR6, CYSLTR2, FAT1, P2RY12, SLC4A4, ACSL5 and GUCY1B3 are higher than the reference value and/or when the expression level of at least one genes selected in AC073172.1, AC092171.4, AC132872.1, AL133351.4, AL391832.3, ALG1L13P, ALPL, AP000640.1, CA4, DGAT2, DHRS13, FBXL13, GALNT14, HOMER3, KAZN, KIAA0319, LINC00963, NFE4, NLRP12, NLRP6, P4HA2, PLB1 and WLS are lower than the reference value.

According to the invention, it will be concluded than the patient will respond to antipsychotic treatment when the expression level of at least one genes selected in C15orf54, TRPC6, CXCR6, CYSLTR2, FAT1, P2RY12, SLC4A4, ACSL5 and GUCY1B3 are lower than the reference value and/or when the expression level of at least one genes selected in AC073172.1, AC092171.4, AC132872.1, AL133351.4, AL391832.3, ALG1L13P, ALPL, AP000640.1, CA4, DGAT2, DHRS13, FBXL13, GALNT14, HOMER3, KAZN, KIAA0319, LINC00963, NFE4, NLRP12, NLRP6, P4HA2, PLB1 and WLS are higher than the reference value.

In another words, the invention relates to a method for predicting antipsychotic treatment response of a patient in need thereof, comprising: i) determining, in a sample obtained from the patient, the expression level of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 and 32 genes selected in the group consisting in AC073172.1, AC092171.4, AC132872.1, ACSL5, AL133351.4, AL391832.3, ALG1L13P, ALPL, AP000640.1, C15orf54, CA4, CXCR6, CYSLTR2, DGAT2, DHRS13, FAT1, FBXL13, GALNT14, GUCY1B3, HOMER3, KAZN, KIAA0319, LINC00963, NFE4, NLRP12, NLRP6, P2RY12, P4HA2, PLB1, SLC4A4, TRPC6, and WLS; ii) comparing the expression of the genes determined at step i) with a reference values and iii) concluding that the patient will not respond to antipsychotic treatment when the expression level determined at step i) is significantly different from the reference value.

In one embodiment, the expression level of all of the gene are determined. Thus, in a particular, the invention relates to a method for predicting antipsychotic treatment response of a patient in need thereof, comprising: i) determining, in a sample obtained from the patient, the expression level of the genes selected in CA4, AC073172.1, NFE4, AP000640.1, C15orf54, GALNT14, KAZN, KIAA0319, TRPC6, AC092171.4, AC132872.1, AL133351.4, AL391832.3, ALG1L13P, ALPL, CXCR6, CYSLTR2, DGAT2, FAT1, FBXL13, LINC00963, NLRP12, NLRP6, P2RY12, P4H42, PLB1, SLC4A4, WLS, ACSL5, GUCY1B3, HOMER3, and DHRS13; ii) comparing the expression of the genes determined at step i) with a reference values and iii) concluding that the patient will not respond to antipsychotic treatment when the expression level determined at step i) is significantly different from the reference value.

In some embodiment, the genes whose expression level is determined in step i) are ACSL5, APLP, CA4, KAZN and KIAA0319.

In some embodiment, the genes whose expression level is determined in step i) are, APLP, CA4, DHRS13, GALNT14, KAZN and HOMER3.

In some embodiment, the genes whose expression level is determined in step i) are, APLP, CA4, DHRS13, DGAT2, WLS and HOMER3.

In some embodiment, the genes whose expression level is determined in step i) are ALPL, CA4, DHRS13, and HOMER3.

In some embodiment, the gene whose expression level is determined in step i) is CA4.

In some embodiment, the gene whose expression level is determined in step i) is DHRS13.

In some embodiment, the gene whose expression level is determined in step i) is HOMER3.

In preferred embodiment, the gene whose expression level is determined in step i) is ALPL.

In some embodiment, the gene whose expression level is determined in step i) is DGAT2.

In some embodiment, the gene whose expression level is determined in step i) is WLS.

In some embodiment, the gene whose expression level is determined in step i) are ALPL and CA4.

In some embodiment, the gene whose expression level is determined in step i) are ALPL, CA4 and DHRS13.

In some embodiment, the gene whose expression level is determined in step i) are ALPL and DHRS13.

In some embodiment, the gene whose expression level is determined in step i) are ALPL, DHRS13 and HOMER3.

In some embodiment, the gene whose expression level is determined in step i) are ALPL and HOMER3.

In some embodiment, the gene whose expression level is determined in step i) are CA4 and HOMER3.

In some embodiment, the gene whose expression level is determined in step i) are CA4 and DHRS13.

In some embodiment, the gene whose expression level is determined in step i) are CA4 HOMER 3 and DHRS13.

In some embodiment, the gene whose expression level is determined in step i) are DHRS13 and HOMER3.

Thus, the invention also refers to a method for predicting antipsychotic treatment response of a patient in need thereof, comprising: i) determining, in a sample obtained from the patient, the expression level of ALPL, CA4, DHRS13 and/or HOMER3; ii) comparing the expression of the genes determined at step i) with a reference values and iii) concluding that the patient will not respond to antipsychotic treatment when the expression level determined at step i) is lower than the reference value.

Thus, the invention also refers to a method for predicting antipsychotic treatment response of a patient in need thereof, comprising: i) determining, in a sample obtained from the patient, the expression level of ALPL, CA4, DHRS13, DGAT2, WLS and/or HOMER3; ii) comparing the expression of the genes determined at step i) with a reference values and iii) concluding that the patient will not respond to antipsychotic treatment when the expression level determined at step i) is lower than the reference value

In some embodiment, the invention refers to a method for predicting antipsychotic treatment response of a patient in need thereof, comprising: i) determining, in a sample obtained from the patient, the expression level of ALPL and at least one gene selected in AC073172.1, AC092171.4, AC132872.1, ACSL5, AL133351.4, AL391832.3, ALG1L13P, AP000640.1, C15orf54, CA4, CXCR6, CYSLTR2, DGAT2, DHRS13, FAT1, FBXL13, GALNT14, GUCY1B3, HOMER3, KAZN, KIAA0319, LINC00963, NFE4, NLRP12, NLRP6, P2RY12, P4HA2, PLB1, SLC4A4, TRPC6, and WLS; ii) comparing the expression of genes determined at step i) with a reference values and iii) concluding that the patient will not respond to antipsychotic treatment when the expression level determined at step i) is significantly different from the reference value.

In some embodiment, the invention refers to a method for predicting antipsychotic treatment response of a patient in need thereof, comprising: i) determining, in a sample obtained from the patient, the expression level of CA4 and at least one gene selected in AC073172.1, AC092171.4, AC132872.1, ACSL5, AL133351.4, AL391832.3, ALG1L13P, ALPL, AP000640.1, C15orf54, CXCR6, CYSLTR2, DGAT2, DHRS13, FAT1, FBXL13, GALNT14, GUCY1B3, HOMER3, KAZN, KIAA0319, LINC00963, NFE4, NLRP12, NLRP6, P2RY12, P4HA2, PLB1, SLC4A4, TRPC6, and WLS; ii) comparing the expression of genes determined at step i) with a reference values and iii) concluding that the patient will not respond to antipsychotic treatment when the expression level determined at step i) is significantly different from the reference value.

In some embodiment, the invention refers to a method for predicting antipsychotic treatment response of a patient in need thereof, comprising: i) determining, in a sample obtained from the patient, the expression level of DHRS13 and at least one gene selected in AC073172.1, AC092171.4, AC132872.1, ACSL5, AL133351.4, AL391832.3, ALG1L13P, ALPL, AP000640.1, C15orf54, CA4, CXCR6, CYSLTR2, DGAT2, FAT1, FBXL13, GALNT14, GUCY1B3, HOMER3, KAZN, KIAA0319, LINC00963, NFE4, NLRP12, NLRP6, P2RY12, P4HA2, PLB1, SLC4A4, TRPC6, and WLS; ii) comparing the expression of genes determined at step i) with a reference values and iii) concluding that the patient will not respond to antipsychotic treatment when the expression level determined at step i) is significantly different from the reference value.

In some embodiment, the invention refers to a method for predicting antipsychotic treatment response of a patient in need thereof, comprising: i) determining, in a sample obtained from the patient, the expression level of HOMER3 and at least one gene selected in AC073172.1, AC092171.4, AC132872.1, ACSL5, AL133351.4, AL391832.3, ALG1L13P, ALPL, AP000640.1, C15orf54, CA4, CXCR6, CYSLTR2, DGAT2, DHRS13FAT1, FBXL13, GALNT14, GUCY1B3, KAZN, KIAA0319, LINC00963, NFE4, NLRP12, NLRP6, P2RY12, P4HA2, PLB1, SLC4A4, TRPC6, and WLS; ii) comparing the expression of genes determined at step i) with a reference values and iii) concluding that the patient will not respond to antipsychotic treatment when the expression level determined at step i) is significantly different than the reference value.

In some embodiment, the invention refers to a method for predicting antipsychotic treatment response of a patient in need thereof, comprising: i) determining, in a sample obtained from the patient, the expression level of DGAT2 and at least one gene selected in AC073172.1, AC092171.4, AC132872.1, ACSL5, AL133351.4, AL391832.3, ALG1L13P, ALPL, AP000640.1, C15orf54, CA4, CXCR6, CYSLTR2, DHRS13, FAT1, FBXL13, GALNT14, GUCY1B3, HOMER3, KAZN, KIAA0319, LINC00963, NFE4, NLRP12, NLRP6, P2RY12, P4HA2, PLB1, SLC4A4, TRPC6, and WLS; ii) comparing the expression of genes determined at step i) with a reference values and iii) concluding that the patient will not respond to antipsychotic treatment when the expression level determined at step i) is significantly different than the reference value.

In some embodiment, the invention refers to a method for predicting antipsychotic treatment response of a patient in need thereof, comprising: i) determining, in a sample obtained from the patient, the expression level of WLS and at least one gene selected in AC073172.1, AC092171.4, AC132872.1, ACSL5, AL133351.4, AL391832.3, ALG1L13P, ALPL, AP000640.1, C15orf54, CA4, CXCR6, CYSLTR2, DGAT2, DHRS13, FAT1, FBXL13, GALNT14, GUCY1B3, HOMER3, KAZN, KIAA0319, LINC00963, NFE4, NLRP12, NLRP6, P2RY12, P4HA2, PLB1, SLC4A4, and TRPC6; ii) comparing the expression of genes determined at step i) with a reference values and iii) concluding that the patient will not respond to antipsychotic treatment when the expression level determined at step i) is significantly different than the reference value.

In some embodiment, the antipsychotic treatment is an amisulpride or olanzapine treatment.

As used herein, the term “patient” refers to any mammal, such as a rodent, a feline, a canine, and a primate. Particularly, in the present invention, the term “patient” refers to a human afflicted with psychosis.

As used herein, the term “psychosis” refers to mental illness, typically characterized by radical changes in personality, impaired functioning, and a distorted or nonexistent sense of objective reality. Psychosis are referenced and classified in Diagnostic and Statistical Manual of Mental Disorders (DSM) published by the American Psychiatric Association (APA) or in ICD-10 Chapter V: Mental and Behavioural Disorders published by the World Health Organization (WHO). Example of psychosis include schizophrenia; schizophreniform disorder; schizoaffective disorder; personality disorder such as schizotypal personality disorder, paranoid personality disorder, schizoid personality disorder and borderline personality disorder; bipolar disorder; sleep deprivation; affective disorders such as major depression, severe depression, depression; brief psychotic disorder; delusional disorder; chronic hallucinatory psychosis; post-traumatic stress disorder; induced delusional disorder; obsessive-compulsive disorder, dissociative disorder; menstrual psychosis; postpartum psychosis; monothematic delusions; myxedematous psychosis; stimulant psychosis; tardive psychosis; shared psychosis. A very large number of medical conditions can cause psychosis, called secondary psychosis. Example of secondary psychosis include disorder causing delirium; neurodevelopmental disorders and chromosomal abnormalities such as velocardiofacial syndrome; neurodegenerative disorders such as Alzheimer' s disease, dementia with Lewy bodies and Parkinson disease; focal neurological disease such as stroke, brain tumor, multiple sclerosis and epilepsy, brain malignancy; infectious syndrome such as viral encephalitis, malaria, syphilis human immunodeficiency virus infection and acquired immune deficiency syndrome; endocrine disease such as hypothyroidism, hyperthyroidism, Cushing's syndrome, hypoparathyroidism and hyperparathyroidism; inborn errors of metabolism such as Succinic semialdehyde dehydrogenase deficiency, porphyria and metachromatic leukodystrophy; nutritional deficiency such as vitamin B₁₂ deficiency; acquired metabolic disorders such as hypocalcemia, hypernatremia, hyponatremia, hypokalemia, hypomagnesemia, hypermagnesemia, hypercalcemia, hypophosphatemia, hypoglycemia and hypoxia; autoimmune disorders such as systemic lupus erythematosus, sarcoidosis, Hashimoto's encephalopathy, anti-NMDA-receptor encephalitis and non-celiac gluten sensitivity; sleep disorders such as narcolespsy; and parasitic diseases such as neurocysticercosis. Various psychoactive substances can cause, exacerbate or precipitate psychosis episode. Drugs commonly alleged to induce psychotic symptoms include alcohol; cannabis; cocaine; amphetamines; cathinones, k-opioid receptor agonist such as enadoline and salvinorin A; NMDA receptor antagonists such as phencyclidine and ketamine.

In some embodiment, the patient is afflicted with first episode psychosis.

In one embodiment, the patient is a patient diagnosed with schizophrenia, schizophreniform disorder or schizoaffective disorder.

In some embodiment, the patient is a medication-naïve patient, i.e a patient who do not have antipsychotic medication before.

In some embodiment, the patient is a patient who has undergone antipsychotic treatment before.

In some embodiment the patient is a patient who has undergone an amisulpride treatment.

In some embodiment, the patient is a patient who has undergone an olanzapine treatment.

As used herein, the term “antipsychotic treatment” refers to psychosis treatment that use one or more antipsychotic agent.

As used herein, the term “antipsychotic agent”, also known as neuroleptics or major tranquillizers, refers to chemical compounds that are effective to manage psychosis. Example of antipsychotic agent include butyrophenones such as benperidol, bromperidol, droperidol, haloperidol, moperone, pipamerone, melperone and timiperone; diphenylbutylpiperidines such as fluspirilene, penfluridol and pimozide; phenotiazines such as acepromazine, chlorpromazine, cyamemazine, dixyrazine, fluphenazine, levomepromazine, mesoridazine, perazine, perphenazine, pipotiazine, prochlorperazine, promazine, promethazine, prothipendyl, thioproperazine, thioridazine trifluoperazine and triflupromazine; thioxanthenes such as chlorprothixene, clopenthixol, flupentixol, thiothixene and zuclopenthixol; benzamides such as sulpiride, sultopride, veralipride, amisulpride; nemonapride, remoxipride and sultopride; tricyclics such as asenapine, olanzapine, quetiapine, zotepine, carpipramine, clocapramine, clorotepine, clotiapine, loxapine and mosapramine; molindone; benzisoaxoles such as lloperidone, lurasidone, paliperidone, paliperidone palmitate, perospirone, risperidone and ziprasidone; phenylpiperazines such as aripiprazole, aripiprazole lauxoril, brexipiparazole and cariprazine; blonanserin; pimavanserin and sertindole. In preferred embodiment the antipsychotic treatement is amilsupride or olanzapine.

As used herein, the term “amisulpride” has its general meaning in the art and refers to 4-amino-N-((1-ethyl-2-pyrrolidinyl)methyl)-5-(etgylsulfonyl)-2-methoxybenzamide, a dopamine D₂ and D₃ receptors antagonist. Its CAS number is 71675-85-9.

As used herein, the term “olanzapine” has its general meaning in the art and refers to 2-méthyl-4-(4-méthyl-1-pipérazinyl)-10H-thiéno[2,3-b][1,5]benzodiazepine, a dopamine D₂ receptors antagonist and serotonin type 2 (5-HT₂) receptors antagonists. Its CAS number is 132539-06-1.

As used herein and according to all aspects of the invention, the term “sample” denotes blood, fresh whole blood, peripheral-blood, peripheral blood mononuclear cell (PBMC), lymp sample. In particular embodiment, the sample is a blood sample, and more particularly peripheral blood mononuclear cell (PBMC).

As used herein, the term “CA4”, for “carbonic anhydrase 4”, refers to gene encoding for a zinc metalloenzyme catalysing the reversible hydration of carbon dioxide and participating in a variety of biological process, including respiration, calcification, acid-base balance, bone resorption, and the formation of aqueous humor, cerebrospinal fluid, saliva, and gastric acid. Its Entrez reference is 762.

As used herein, the term “AC073172.1” refers to a novel transcript located on chromosome 11:15,571,819-15,622,403.

As used herein, the term “NFE4”, for “nuclear factor, erythroid 4”, refers to gene encoding for an erythroid-specific protein which, with the ubiquitous transcription factor CP2, form the stage selector protein (SSP) complex involved in preferential expression of the gamma-globin genes in fetal erythroid cells. Its Entrez reference is 58160.

As used herein, the term “AP000640.1” refers to a novel transcript located on chromosome 11:59,752,578-59,754,975.

As used herein, the term “C15orf54”, for “chromosome 15 open reading frame 54”, refers to an RNA gene affiliated with non-coding RNA class. Its Entrez reference is 400360.

As used herein, the term “GALNT14”, for “polypeptide GalNAc transferase 14”, refers to a gene encoding a Golgi protein which is a member of the polypeptide N-acetylgalactosaminyltransferase protein family. Its Entrez reference is 79623.

As used herein, the term “KAZN”, for “kazrin”, refers to a gene encoding a protein that plays a role in desmosome assembly, cell adhesion, cytoskeletal organization, and epidermal differentiation. Its Entrez reference is 23254.

As used herein, the term “KIAA0319”, for “dyslexia-associated protein”, refers to a gene encoding a transmembrane protein that contains a large extracellular domain with multiple polycystic kidney disease (PKD) domains. Its Entrez reference is 9856.

As used herein, the term “TRPC6”, for “transient receptor potential cation channel subfamily C member 6”, refers to a gene encoding a receptor-activated calcium channel in the cell membrane. Its Entrez reference is 7225.

As used herein, the term “AC092171.4” refers to a novel transcript located on chromosome 7:5,425,770-5,426,401.

As used herein, the term “AC132872.1” refers to a novel transcript located on chromosome 17:82,293,716-82,294,910.

As used herein, the tem “AL133351.4” refers to transcript located on chromosome 6:3,033,183-3,033,288.

As used herein, the term “AL391832.3” refers to a novel transcript located on chromosome 1:234,979,647-234,980,804.

As used herein, the term “ALG1L13P” refers to an asparagine-linked glycosylation 1-like 13 pseudogene located on chromosome 8:8,236,003-8,244,667. Its Entrez reference is 106479038.

As used herein, the term “ALPL”, for “alkaline phosphatase, biomineralization associated” refers to a gene encoding a membrane bound glycosylated enzyme, member of the alkaline phosphatase family of proteins, which is not expressed in any particular tissue and is, therefore, referred to as the tissue-nonspecific form of the enzyme. Its Entrez reference is 249.

As used herein, the term “CXCR6” refers to a gene encoding C-X-C chemokine receptor type 6, also known as CD186. Its Entrez reference is 10663.

As used herein, the term “CYSLTR2”, for “cysteinyl leukotriene receptor 2”, refers to a gene encoding cysteinyl leukotrienes which are important mediators of human bronchial asthma. Its Entrez reference is 57105.

As used herein, the term “DGAT2”, for “diacylglycerol O-acyltransferase 2”, refers to a gene encoding one of two enzymes which catalyzes the final reaction in the synthesis of triglycerides in which diacylglycerol is covalently bound to long chain fatty acyl-CoAs. Its Entrez reference is 84649.

As used herein, the term “FAT1”, for “FAT atypical cadherin 1”, refers to a gene encoding a member of the cadherin superfamily, a group of integral membrane proteins characterized by the presence of cadherin-type repeats. Its Entrez reference is 2195.

As used herein, the term “FBXL13” refers to a gene encoding f-box and leucine rich repeat protein 1 which form SCF complexes with SKP1 and cullin and act as protein-ubiquitin ligases. Its Entrez reference is 222235.

As used herein, the term “LINC00963”, for “long intergenic non-protein coding RNA 963”, refers to an RNA gene and is affiliated with the non-coding RNA class. Its Entrez reference is 100506190.

As used herein, the term “NLRP12”, for “NLR family pyrin domain containing 12”, refers to a gene encoding a member of the CATERPILLER family of cytoplasmic proteins which functions as an attenuating factor of inflammation by suppressing inflammatory responses in activated monocytes. Its Entrez reference is 91662.

As used herein, the term “NLRP6”, for “NLR family pyrin domain containing 6”, refers to a gene encoding a protein which binds arginine-vasopressin and may be involved in the arginine-vasopressin-mediated regulation of renal salt-water balance. Its Entrez reference is 171389.

As used herein, the term “P2RY12”, for “purinergic receptor P2Y12”, refers to a gene encoding a G-protein coupled receptors involved in platelet aggregation. Its Entrez reference is 64805.

As used herein, the term “P4HA2”, for “prolyl 4-hydroxylase subunit alpha 2”, refers to a gene encoding a component of prolyl 4-hydroxylase, a key enzyme in collagen synthesis composed of two identical alpha subunits and two beta subunits. Its Entrez reference is 8974.

As used herein, the term “PLB1”, for “phospholipase B1”, refers to a gene encoding membrane-associated phospholipase that displays lysophospholipase and phospholipase A2 activities through removal of sn-1 and sn-2 fatty acids of glycerophospholipids. Its Entrez reference is 151056.

As used herein, the term “SLC4A4”, for “solute carrier family 4 member 4”, refers to a gene encoding a sodium bicarbonate cotransporter (NBC) involved in the regulation of bicarbonate secretion and absorption and intracellular pH. Its Entrez reference is 8671.

As used herein, the term “WLS”, for “Wntless Wnt ligand secretion mediator”, refers to a gene encoding a receptor for Wnt protein. Its Entrez reference is 79971.

As used herein, the term “ACSL5”, for “acyl-coA synthetase long chain family member 5”, refers to a gene encoding an isozyme of the long-chain fatty-acid-coenzyme A ligase family which convert free long-chain fatty acids into fatty acyl-CoA esters, and thereby play a key role in lipid biosynthesis and fatty acid degradation. Its Entrez reference is 51703.

As used herein, the term “GUCY1B3”, for “guanylate cyclase 1 soluble subunit beta 1”, refers to a gene encoding the beta subunit of the soluble guanylate cyclase (sGC), which catalyzes the conversion of GTP (guanosine triphosphate) to cGMP (cyclic guanosine monophosphate). Its Entrez reference is 2983.

As used herein, the term “HOMER3”, for “homer scaffold protein 3”, refers to a gene encoding a member of the HOMER family of postsynaptic density scaffolding proteins that share a similar domain structure consisting of an N-terminal Enabled/vasodilator-stimulated phosphoprotein homology 1 domain which mediates protein-protein interactions. Its Entrez reference is 9454.

As used herein, the term “DHRS13”, for “dehydrogenase/reductase 13”, refers to a gene encoding a putative oxidoreductase. Its Entrez reference is 147015.

As used herein, a “reference value” can be a “threshold value” or a “cut-off value”. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. Preferably, the person skilled in the art may compare the gene expression level (obtained according to the method of the invention) with a defined threshold value

Each reference (“cut-off”) value for the genes' expression may be predetermined by carrying out a method comprising the steps of

a) providing a collection of samples from patients suffering of psychosis (after diagnosis of psychosis for example);

b) determining the expression level of the genes for each sample contained in the collection provided at step a)

c) ranking the samples according to said gene expression level determined and determining a threshold value above which the expression level is said to be “high” and below which the expression level is said to be “low”;

d) quantitatively defining the threshold/cut-off/reference value by determining the number of copies of the said gene corresponding to the threshold/cut-off/reference value; to be done by constructing a calibration curve using known input quantities of cDNA or protein for the said gene;

e) classifying said samples in pairs of subsets of increasing, respectively decreasing, number of members ranked according to their expression level,

f) providing, for each sample provided at step a), information relating to the actual treatment outcome for the corresponding patient (i.e. good or poor responders after 4 weeks of treatment with amisulpride);

g) for each pair of subsets of samples, obtaining a cluster of expression fold changes using Euclidian distance.

h) for each pair of subsets of samples calculating the statistical significance (p value) between both subsets

i) selecting as reference value for the expression level, the value of expression level for which the p value is the smallest.

For example the expression level of the genes has been assessed for 100 samples from 100 patients. The 100 samples are ranked according to their expression level. Sample 1 has the highest expression level and sample 100 has the lowest expression level. A first grouping provides two subsets: on one side sample Nr 1 and on the other side the 99 other samples. The next grouping provides on one side samples 1 and 2 and on the other side the 98 remaining samples etc., until the last grouping: on one side samples 1 to 99 and on the other side sample Nr 100. According to the information relating to the actual treatment outcome for the corresponding patient, Euclidian distance are prepared for each of the 99 groups of two subsets. Also for each of the 99 groups, the p value between both subsets was calculated.

The reference value is selected such as the discrimination based on the criterion of the minimum p value is the strongest. In other terms, the expression level corresponding to the boundary between both subsets for which the p value is minimum is considered as the reference value. It should be noted that the reference value is not necessarily the median value of expression levels. In routine work, the reference value (cut-off value) may be used in the present method to discriminate good and poor antipsychotic responders.

Euclidian distances are commonly used to measure the dissimilarity between expression profiles with regard to the signature genes and are well known by the person skilled in the art. The man skilled in the art also understands that the same technique of assessment of the expression level of a gene should of course be used for obtaining the reference value and thereafter for assessment of the expression level of a gene of a patient subjected to the method of the invention.

Such predetermined reference values of expression level may be determined for any gene defined above.

Measuring the expression level of the genes listed above can be done by measuring the gene expression level of these genes and can be performed by a variety of techniques well known in the art.

Typically, the expression level of a gene may be determined by determining the quantity of mRNA. Methods for determining the quantity of mRNA are well known in the art. For example, the nucleic acid contained in the samples (e.g., cell or tissue prepared from the patient) is first extracted according to standard methods, for example using lytic enzymes or chemical solutions or extracted by nucleic-acid-binding resins following the manufacturer's instructions. The extracted mRNA is then detected by hybridization (e. g., Northern blot analysis, in situ hybridization) and/or amplification (e.g., RT-PCR).

Other methods of Amplification include ligase chain reaction (LCR), transcription-mediated amplification (TMA), strand displacement amplification (SDA) and nucleic acid sequence-based amplification (NASBA).

Nucleic acids having at least 10 nucleotides and exhibiting sequence complementarity or homology to the mRNA of interest herein find utility as hybridization probes or amplification primers. It is understood that such nucleic acids need not be identical, but are typically at least about 80% identical to the homologous region of comparable size, more preferably 85% identical and even more preferably 90-95% identical. In certain embodiments, it will be advantageous to use nucleic acids in combination with appropriate means, such as a detectable label, for detecting hybridization.

Typically, the nucleic acid probes include one or more labels, for example to permit detection of a target nucleic acid molecule using the disclosed probes. In various applications, such as in situ hybridization procedures, a nucleic acid probe includes a label (e.g., a detectable label). A “detectable label” is a molecule or material that can be used to produce a detectable signal that indicates the presence or concentration of the probe (particularly the bound or hybridized probe) in a sample. Thus, a labeled nucleic acid molecule provides an indicator of the presence or concentration of a target nucleic acid sequence (e.g., genomic target nucleic acid sequence) (to which the labeled uniquely specific nucleic acid molecule is bound or hybridized) in a sample. A label associated with one or more nucleic acid molecules (such as a probe generated by the disclosed methods) can be detected either directly or indirectly. A label can be detected by any known or yet to be discovered mechanism including absorption, emission and/or scattering of a photon (including radio frequency, microwave frequency, infrared frequency, visible frequency and ultra-violet frequency photons). Detectable labels include colored, fluorescent, phosphorescent and luminescent molecules and materials, catalysts (such as enzymes) that convert one substance into another substance to provide a detectable difference (such as by converting a colorless substance into a colored substance or vice versa, or by producing a precipitate or increasing sample turbidity), haptens that can be detected by antibody binding interactions, and paramagnetic and magnetic molecules or materials.

Particular examples of detectable labels include fluorescent molecules (or fluorochromes). Numerous fluorochromes are known to those of skill in the art, and can be selected, for example from Life Technologies (formerly Invitrogen), e.g., see, The Handbook—A Guide to Fluorescent Probes and Labeling Technologies). Examples of particular fluorophores that can be attached (for example, chemically conjugated) to a nucleic acid molecule (such as a uniquely specific binding region) are provided in U.S. Pat. No. 5,866,366 to Nazarenko et al., such as 4-acetamido-4′-isothiocyanatostilbene-2,2′ disulfonic acid, acridine and derivatives such as acridine and acridine isothiocyanate, 5-(2′-aminoethyl) aminonaphthalene-1-sulfonic acid (EDANS), 4-amino-N-[3 vinylsulfonyl)phenyl]naphthalimide-3,5 disulfonate (Lucifer Yellow VS), N-(4-anilino-1-naphthyl)maleimide, antl1ranilamide, Brilliant Yellow, coumarin and derivatives such as coumarin, 7-amino-4-methylcoumarin (AMC, Coumarin 120), 7-amino-4-trifluoromethylcouluarin (Coumarin 151); cyanosine; 4′,6-diarninidino-2-phenylindole (DAPI); 5′,5″dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulforlic acid; 5-[dimethylamino] naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6dicl1lorotriazin-2-yDarninofluorescein (DTAF), 2′7′dimethoxy-4′5′-dichloro-6-carboxyfluorescein (JOE), fluorescein, fluorescein isothiocyanate (FITC), and QFITC Q(RITC); 2′,7′-difluorofluorescein (OREGON GREEN®); fluorescamine; IR144; IR1446; Malachite Green isothiocyanate; 4-methylumbelliferone; ortho cresolphthalein; nitrotyrosine; pararosaniline; Phenol Red; B-phycoerythrin; o-phthaldialdehyde; pyrene and derivatives such as pyrene, pyrene butyrate and succinimidyl 1-pyrene butyrate; Reactive Red 4 (Cibacron Brilliant Red 3B-A); rhodamine and derivatives such as 6-carboxy-X-rhodamine (ROX), 6-carboxyrhodamine (R6G), lissamine rhodamine B sulfonyl chloride, rhodamine (Rhod), rhodamine B, rhodamine 123, rhodamine X isothiocyanate, rhodamine green, sulforhodamine B, sulforhodamine 101 and sulfonyl chloride derivative of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; rosolic acid and terbium chelate derivatives. Other suitable fluorophores include thiol-reactive europium chelates which emit at approximately 617 mn (Heyduk and Heyduk, Analyt. Biochem. 248:216-27, 1997; J. Biol. Chem. 274:3315-22, 1999), as well as GFP, Lissamine™, diethylaminocoumarin, fluorescein chlorotriazinyl, naphthofluorescein, 4,7-dichlororhodamine and xanthene (as described in U.S. Pat. No. 5,800,996 to Lee et al.) and derivatives thereof. Other fluorophores known to those skilled in the art can also be used, for example those available from Life Technologies (Invitrogen; Molecular Probes (Eugene, Oreg.)) and including the ALEXA FLUOR® series of dyes (for example, as described in U.S. Pat. Nos. 5,696,157, 6,130,101 and 6,716,979), the BODIPY series of dyes (dipyrrometheneboron difluoride dyes, for example as described in U.S. Pat. Nos. 4,774,339, 5,187,288, 5,248,782, 5,274,113, 5,338,854, 5,451,663 and 5,433,896), Cascade Blue (an amine reactive derivative of the sulfonated pyrene described in U.S. Pat. No. 5,132,432) and Marina Blue (U.S. Pat. No. 5,830,912).

In addition to the fluorochromes described above, a fluorescent label can be a fluorescent nanoparticle, such as a semiconductor nanocrystal, e.g., a QUANTUM DOT™ (obtained, for example, from Life Technologies (QuantumDot Corp, Invitrogen Nanocrystal Technologies, Eugene, Oreg.); see also, U.S. Pat. Nos. 6,815,064; 6,682,596; and 6,649,138). Semiconductor nanocrystals are microscopic particles having size-dependent optical and/or electrical properties. When semiconductor nanocrystals are illuminated with a primary energy source, a secondary emission of energy occurs of a frequency that corresponds to the handgap of the semiconductor material used in the semiconductor nanocrystal. This emission can be detected as colored light of a specific wavelength or fluorescence. Semiconductor nanocrystals with different spectral characteristics are described in e.g., U.S. Pat. No. 6,602,671. Semiconductor nanocrystals that can be coupled to a variety of biological molecules (including dNTPs and/or nucleic acids) or substrates by techniques described in, for example, Bruchez et al., Science 281 :20132016, 1998; Chan et al., Science 281:2016-2018, 1998; and U.S. Pat. No. 6,274,323. Formation of semiconductor nanocrystals of various compositions are disclosed in, e.g., U.S. Pat. Nos. 6,927,069; 6,914,256; 6,855,202; 6,709,929; 6,689,338; 6,500,622; 6,306,736; 6,225,198; 6,207,392; 6,114,038; 6,048,616; 5,990,479; 5,690,807; 5,571,018; 5,505,928; 5,262,357 and in U.S. Patent Publication No. 2003/0165951 as well as PCT Publication No. 99/26299 (published May 27, 1999). Separate populations of semiconductor nanocrystals can be produced that are identifiable based on their different spectral characteristics. For example, semiconductor nanocrystals can be produced that emit light of different colors hased on their composition, size or size and composition. For example, quantum dots that emit light at different wavelengths based on size (565 mn, 655 mn, 705 mn, or 800 mn emission wavelengths), which are suitable as fluorescent labels in the probes disclosed herein are available from Life Technologies (Carlshad, Calif.).

Additional labels include, for example, radioisotopes (such as 3 H), metal chelates such as DOTA and DPTA chelates of radioactive or paramagnetic metal ions like Gd3+, and liposomes.

Detectable labels that can be used with nucleic acid molecules also include enzymes, for example horseradish peroxidase, alkaline phosphatase, acid phosphatase, glucose oxidase, beta-galactosidase, beta-glucuronidase, or beta-lactamase.

Alternatively, an enzyme can be used in a metallographic detection scheme. For example, silver in situ hyhridization (SISH) procedures involve metallographic detection schemes for identification and localization of a hybridized genomic target nucleic acid sequence. Metallographic detection methods include using an enzyme, such as alkaline phosphatase, in combination with a water-soluble metal ion and a redox-inactive substrate of the enzyme. The substrate is converted to a redox-active agent by the enzyme, and the redoxactive agent reduces the metal ion, causing it to form a detectable precipitate. (See, for example, U.S. Patent Application Publication No. 2005/0100976, PCT Publication No. 2005/003777 and U.S. Patent Application Publication No. 2004/0265922). Metallographic detection methods also include using an oxido-reductase enzyme (such as horseradish peroxidase) along with a water soluble metal ion, an oxidizing agent and a reducing agent, again to form a detectable precipitate. (See, for example, U.S. Pat. No. 6,670,113).

Probes made using the disclosed methods can be used for nucleic acid detection, such as ISH procedures (for example, fluorescence in situ hybridization (FISH), chromogenic in situ hybridization (CISH) and silver in situ hybridization (SISH)) or comparative genomic hybridization (CGH).

In situ hybridization (ISH) involves contacting a sample containing target nucleic acid sequence (e.g., genomic target nucleic acid sequence) in the context of a metaphase or interphase chromosome preparation (such as a cell or tissue sample mounted on a slide) with a labeled probe specifically hybridizable or specific for the target nucleic acid sequence (e.g., genomic target nucleic acid sequence). The slides are optionally pretreated, e.g., to remove paraffin or other materials that can interfere with uniform hybridization. The sample and the probe are both treated, for example by heating to denature the double stranded nucleic acids. The probe (formulated in a suitable hybridization buffer) and the sample are combined, under conditions and for sufficient time to permit hybridization to occur (typically to reach equilibrium). The chromosome preparation is washed to remove excess probe, and detection of specific labeling of the chromosome target is performed using standard techniques.

For example, a biotinylated probe can be detected using fluorescein-labeled avidin or avidin-alkaline phosphatase. For fluorochrome detection, the fluorochrome can be detected directly, or the samples can be incubated, for example, with fluorescein isothiocyanate (FITC)-conjugated avidin. Amplification of the FITC signal can be effected, if necessary, by incubation with biotin-conjugated goat antiavidin antibodies, washing and a second incubation with FITC-conjugated avidin. For detection by enzyme activity, samples can be incubated, for example, with streptavidin, washed, incubated with biotin-conjugated alkaline phosphatase, washed again and pre-equilibrated (e.g., in alkaline phosphatase (AP) buffer). For a general description of in situ hybridization procedures, see, e.g., U.S. Pat. No. 4,888,278.

Numerous procedures for FISH, CISH, and SISH are known in the art. For example, procedures for performing FISH are described in U.S. Pat. Nos. 5,447,841; 5,472,842; and 5,427,932; and for example, in Pir1kel et al., Proc. Natl. Acad. Sci. 83:2934-2938, 1986; Pinkel et al., Proc. Natl. Acad. Sci. 85:9138-9142, 1988; and Lichter et al., Proc. Natl. Acad. Sci. 85:9664-9668, 1988. CISH is described in, e.g., Tanner et al., Am. 1. Pathol. 157:1467-1472, 2000 and U.S. Pat. No. 6,942,970. Additional detection methods are provided in U.S. Pat. No. 6,280,929.

Numerous reagents and detection schemes can be employed in conjunction with FISH, CISH, and SISH procedures to improve sensitivity, resolution, or other desirable properties. As discussed above probes labeled with fluorophores (including fluorescent dyes and QUANTUM DOTS®) can be directly optically detected when performing FISH. Alternatively, the probe can be labeled with a nonfluorescent molecule, such as a hapten (such as the following non-limiting examples: biotin, digoxigenin, DNP, and various oxazoles, pyrrazoles, thiazoles, nitroaryls, benzofurazans, triterpenes, ureas, thioureas, rotenones, coumarin, courmarin-based compounds, Podophyllotoxin, Podophyllotoxin-based compounds, and combinations thereof), ligand or other indirectly detectable moiety. Probes labeled with such non-fluorescent molecules (and the target nucleic acid sequences to which they bind) can then be detected by contacting the sample (e.g., the cell or tissue sample to which the probe is bound) with a labeled detection reagent, such as an antibody (or receptor, or other specific binding partner) specific for the chosen hapten or ligand. The detection reagent can be labeled with a fluorophore (e.g., QUANTUM DOT®) or with another indirectly detectable moiety, or can be contacted with one or more additional specific binding agents (e.g., secondary or specific antibodies), which can be labeled with a fluorophore.

In other examples, the probe, or specific binding agent (such as an antibody, e.g., a primary antibody, receptor or other binding agent) is labeled with an enzyme that is capable of converting a fluorogenic or chromogenic composition into a detectable fluorescent, colored or otherwise detectable signal (e.g., as in deposition of detectable metal particles in SISH). As indicated above, the enzyme can be attached directly or indirectly via a linker to the relevant probe or detection reagent. Examples of suitable reagents (e.g., binding reagents) and chemistries (e.g., linker and attachment chemistries) are described in U.S. Patent Application Publication Nos. 2006/0246524; 2006/0246523, and 2007/0117153.

It will be appreciated by those of skill in the art that by appropriately selecting labelled probe-specific binding agent pairs, multiplex detection schemes can be produced to facilitate detection of multiple target nucleic acid sequences (e.g., genomic target nucleic acid sequences) in a single assay (e.g., on a single cell or tissue sample or on more than one cell or tissue sample). For example, a first probe that corresponds to a first target sequence can be labelled with a first hapten, such as biotin, while a second probe that corresponds to a second target sequence can be labelled with a second hapten, such as DNP. Following exposure of the sample to the probes, the bound probes can be detected by contacting the sample with a first specific binding agent (in this case avidin labelled with a first fluorophore, for example, a first spectrally distinct QUANTUM DOT®, e.g., that emits at 585 mn) and a second specific binding agent (in this case an anti-DNP antibody, or antibody fragment, labelled with a second fluorophore (for example, a second spectrally distinct QUANTUM DOT®, e.g., that emits at 705 mn). Additional probes/binding agent pairs can be added to the multiplex detection scheme using other spectrally distinct fluorophores. Numerous variations of direct, and indirect (one step, two step or more) can be envisioned, all of which are suitable in the context of the disclosed probes and assays.

Probes typically comprise single-stranded nucleic acids of between 10 to 1000 nucleotides in length, for instance of between 10 and 800, more preferably of between 15 and 700, typically of between 20 and 500. Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they preferably hybridize under high stringency hybridization conditions (corresponding to the highest melting temperature Tm, e.g., 50% formamide, 5x or 6x SCC. SCC is a 0.15 M NaCl, 0.015 M Na-citrate).

The nucleic acid primers or probes used in the above amplification and detection method may be assembled as a kit. Such a kit includes consensus primers and molecular probes. A preferred kit also includes the components necessary to determine if amplification has occurred. The kit may also include, for example, PCR buffers and enzymes; positive control sequences, reaction control primers; and instructions for amplifying and detecting the specific sequences.

In a particular embodiment, the methods of the invention comprise the steps of providing total RNAs extracted from cumulus cells and subjecting the RNAs to amplification and hybridization to specific probes, more particularly by means of a quantitative or semi-quantitative RT-PCR.

In another preferred embodiment, the expression level is determined by DNA chip analysis. Such DNA chip or nucleic acid microarray consists of different nucleic acid probes that are chemically attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the expression level, a sample from a test subject, optionally first subjected to a reverse transcription, is labelled and contacted with the microarray in hybridization conditions, leading to the formation of complexes between target nucleic acids that are complementary to probe sequences attached to the microarray surface. The labelled hybridized complexes are then detected and can be quantified or semi-quantified. Labelling may be achieved by various methods, e.g. by using radioactive or fluorescent labelling. Many variants of the microarray hybridization technology are available to the man skilled in the art (see e.g. the review by Hoheisel, Nature Reviews, Genetics, 2006, 7:200-210).

In another embodiment, the expression level is determined by metabolic imaging (see for example Yamashita T et al., Hepatology 2014, 60:1674-1685 or Ueno A et al., Journal of hepatology 2014, 61:1080-1087).

Expression level of a gene may be expressed as absolute expression level or normalized expression level. Typically, expression levels are normalized by correcting the absolute expression level of a gene by comparing its expression to the expression of a gene that is not a relevant for determining the response of antipsychotic treatment, e.g., a housekeeping gene that is constitutively expressed. Suitable genes for normalization include housekeeping genes such as the actin gene ACTB, ribosomal 18S gene, GUSB, PGK1, TFRC, GAPDH, TBP and ABL1. This normalization allows the comparison of the expression level in one sample, e.g., a patient sample, to another sample, or between samples from different sources.

The inventors found that combining the gene-expression profiling with clinical data (such as ancestry, the duration of untreated psychosis; the positive PANSS score, the sex and the age), allow to predict treatment outcome with a predictive value of 93.8%. Indeed they compared good and poor responders using the same function and a statistical model adjusted for age, sex, ancestry, DUP, positive PANSS score of patients at inclusion and centre in which individuals had been recruited, these features either having been shown to be significantly different between future good and poor responders, these features either having been shown to be significantly different between future good and poor responders or being putative confounding factors. Thus the calculation of a logistic regression and the calculation of the optimal threshold value can be done to distinguish the patients that will respond to antipsychotic treatment.

Accordingly, in some embodiment, clinical data can be combined with gene expression to improve power to predict antipsychotic treatment response.

Accordingly, in another aspect, the invention relates to a method for predicting antipsychotic response of a patient suffering from psychosis episode comprising the steps of: i) determining, in a sample obtained from the patient, the expression level of at least one genes selected in the group consisting in AC073172.1, AC092171.4, AC132872.1, ACSL5, AL133351.4, AL391832.3, ALG1L13P, ALPL, AP000640.1, C15orf54, CA4, CXCR6, CYSLTR2, DGAT2, DHRS13, FAT1, FBXL13, GALNT14, GUCY1B3, HOMER3, KAZN, KIAA0319, LINC00963, NFE4, NLRP12, NLRP6, P2RY12, P4HA2, PLB1, SLC4A4, TRPC6, and WLS; ii) comparing the expression of the genes determined at step i) with a reference values, iii) regarding the clinical data of said patient, iv) calculating the clinical data score of the patient, using the clinical data from step iii), according to the logistic regression defined from a learning antipsychotic responders cohort and iii) concluding that the patient will not respond to antipsychotic treatment when the expression level determined at step i) is significantly different from the reference value and when clinical data score is lower from the optimal threshold.

In some embodiment, the genes whose expression level is determined in step i) are, APLP, CA4, DHRS13, DGAT2, WLS and/or HOMER3.

In some embodiment, the genes whose expression level is determined in step i) are ALPL, CA4, DHRS13, and/or HOMER3.

In some embodiment, the genes whose expression level is determined in step i) is ALPL.

In some embodiment, the clinical data combined with gene expression are the age, the ancestry, the duration of untreated psychosis (DUP), the sex and/or the positive PANSS score of the patient.

In some embodiment, the clinical data combined with gene expression are the age, the duration of untreated psychosis (DUP), the sex and/or the positive PANSS score of the patient.

In some embodiment, the clinical data combined with gene expression are the age, the duration of untreated psychosis (DUP), and/or the positive PANSS score of the patients.

Thus, the invention relates to a method for predicting antipsychotic response of a patient suffering from psychosis episode comprising the steps of: i) determining, in a sample obtained from the patient, the expression level of at least one genes selected in the group consisting in ALPL, CA4, DGAT2, DHRS13, HOMER3 and/or WLS; ii) comparing the expression of the genes determined at step i) with a reference values, iii) regarding the clinical data of said patient wherein the clinical are the age, the duration of untreated psychosis (DUP), the sex and/or the positive PANSS score of said patient, iv) calculating the clinical data score of the patient, using the clinical data from step iii), according to the logistic regression defined from a learning antipsychotic responders cohort and iii) concluding that the patient will not respond to antipsychotic treatment when the expression level determined at step i) is lower from the reference value and when clinical data score is lower from the optimal threshold.

In other words, the invention relates to a method for predicting antipsychotic response of a patient suffering from psychosis episode comprising the steps of: i) determining, in a sample obtained from the patient, the expression level of at least one genes selected in the group consisting in ALPL, CA4, DGAT2, DHRS13, HOMER3 and/or WLS; ii) comparing the expression of the genes determined at step i) with a reference values, iii) regarding the clinical data of said patient wherein the clinical are the age, the duration of untreated psychosis (DUP), the sex and/or the positive PANSS score of said patient, iv) calculating the clinical data score of the patient, using the clinical data from step iii), according to the logistic regression defined from a learning antipsychotic responders cohort and iii) concluding that the patient will respond to antipsychotic treatment when the expression level determined at step i) is higher from the reference value and when clinical data score is higher from the optimal threshold

As used herein the term “clinical data score” denotes the score of a given patient that is calculated with the clinical data using the formula of the logistic regression previously determined from good or poor antipsychotic treatment responder cohort. This score represents the probability of a given patient to respond to antipsychotic treatment based on its clinical data

As used herein, the term “optimal threshold” correspond to the threshold value of the clinical data score that will split the patients in 2 groups: clinical data score above the threshold will corresponds to the group of good responder, namely patients that will respond to the antipsychotic treatment, clinical data score below the threshold will corresponds to the group of poor responder patients. This threshold value of the clinical score is qualified of optimal because it is calculated to obtain the value of clinical data score that will give the best accuracy of prediction with the cohort.

As used herein, the term “PANSS” or “Positive and negative syndrome scale” is well known in the art and refers to a medical scale used for measuring symptom severity of patients with schizophrenia. It was published in 1987 by Stanley Kay, Lewis Opler, and Abraham Fiszbein and is now widely used in the study of antipsychotic therapy. To assess a patient using PANSS, a clinical interview is conducted. The patient is then rated from 1 to 7 on 30 different symptoms (referred as items). The positive PANSS score refers to the sum of 7 items (delusions, conceptual disorganization, hallucination, excitement, grandiosity, suspiciousness and hostility.

As used herein, the term “duration of untreated psychosis” or “DUP” refers the time from manifestation of the first psychotic symptom to initiation of adequate treatment.

As used herein, the term “ancestry” refers to genetic ancestry, namely the genetic architecture of genome variation between populations. Herein we distinguished between individual with a European origin from those with a non-European origin, as defined according to the HapMap populations.

In a second aspect, the invention relates to a method for monitoring amisulpride or olanzapine antipsychotic treatment in patient suffering from psychosis episode comprising the steps of i) measuring in a sample obtained from said patients the expression level of at least one gene selected from the group consisting of AC073172.1, AC092171.4, AC132872.1, ACSL5, AL133351.4, AL391832.3, ALG1L13P, ALPL, AP000640.1, C15orf54, CA4, CXCR6, CYSLTR2, DGAT2, DHRS13, FAT1, FBXL13, GALNT14, GUCY1B3, HOMER3, KAZN, KIAA0319, LINC00963, NFE4, NLRP12, NLRP6, P2RY12, P4HA2, PLB1, SLC4A4, TRPC6, and WLS; ii) comparing the expression level measured at step i) with a reference value, and iii) administering a therapeutically effective amount of clozapine when the gene level determined at step i) is significantly different from the reference value.

In one embodiment, the expression level of all of the gene are determined.

In preferred embodiment, the gene whose expression level is determined in step i) ALPL, CA4, DHRS13, or HOMER3 alone.

In some embodiment, the gene whose expression level is determined in step i) is ALPL, CA4, DHRS13, or HOMER3 and at least one gene selected in AC073172.1, AC092171.4, AC132872.1, ACSL5, AL133351.4, AL391832.3, ALG1L13P, ALPL, AP000640.1, C15orf54, CA4, CXCR6, CYSLTR2, DGAT2, DHRS13, FAT1, FBXL13, GALNT14, GUCY1B3, HOMER3, KAZN, KIAA0319, LINC00963, NFE4, NLRP12, NLRP6, P2RY12, P4HA2, PLB1, SLC4A4, TRPC6, and WLS.

In some embodiment, the gene whose expression level is determined in step i) is ALPL, CA4, DHRS13, HOMER3, DGAT2 or WLS and at least one gene selected in AC073172.1, AC092171.4, AC132872.1, ACSL5, AL133351.4, AL391832.3, ALG1L13P, ALPL, AP000640.1, C15orf54, CA4, CXCR6, CYSLTR2, DGAT2, DHRS13, FAT1, FBXL13, GALNT14, GUCY1B3, HOMER3, KAZN, KIAA0319, LINC00963, NFE4, NLRP12, NLRP6, P2RY12, P4HA2, PLB1, SLC4A4, TRPC6, and WLS.

The invention also relates to a method for monitoring amisulpride or olanzapine antipsychotic treatment in patient suffering from psychosis episode comprising the steps of i) measuring in a sample obtained from said patients the expression level of 1, 2, 3 4, 5 or 6 gene selected from the group consisting of ALPL, CA4, DGAT2, DHRS13, HOMER3, and WLS; ii) comparing the expression level measured at step i) with a reference value, and iii) administering a therapeutically effective amount of clozapine when the gene level determined at step i) is lower than the reference value.

In other word, the invention relates to a method of treating psychosis episode in patient in need thereof comprising the step of i) determining if the patient will respond to antipsychotic drug according to the method of the invention and ii) administering a therapeutically effective amount of clozapine when the patient is determined as a non-responder of antipsychotic drug.

In some embodiment, the antispsychotic drug is amisulpride or olanzapine.

Thus, the invention relates a method of treating psychosis episode comprising the step of i) determining if the patient will respond to amisulpride or olanzapine according to the method of the invention and ii) administering a therapeutically effective amount of clozapine when the patient is determined as a non-responder of amisulpride or olanzapine.

In other word, the invention refers to clozapine for use in the treatment of psychosis episode comprising the step of i) determining if the patient will respond to amisulpride or olanzapine according to the method of the invention and ii) administering a therapeutically effective amount of clozapine when the patient is determined as a non-responder of amisulpride or olanzapine.

Thus, the invention relates a method of treating psychosis episode comprising the steps of i) measuring in a sample obtained from said patients the expression level of 1, 2, 3 4, 5 or 6 gene selected from the group consisting of ALPL, CA4, DGAT2, DHRS13, HOMER3, and WLS; ii) comparing the expression level measured at step i) with a reference value, and iii) administering a therapeutically effective amount of clozapine when the gene level determined at step i) is lower than the reference value

As used herein, the term “clozapine” has its general meaning in the art and refers to 8-chloro-11-(4-méthylpipérazin-1-yl)-5H-dibenzo[b,e][1,4]diazépine, an atypical antipsychotic drug binding to serotonin as well as dopamine receptors. Its CAS number is 5786-21-0.

As used herein, the term “treatment” or “treating” refer to both prophylactic or preventive treatment as well as curative or disease modifying treatment, including treatment of subjects at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, a “therapeutically effective amount” is intended for a minimal amount of active agent which is necessary to impart therapeutic benefit to a patient. For example, a “therapeutically effective amount of clozapine” to a patient is an amount of clozapine that induces, ameliorates or causes an improvement in the pathological symptoms, disease progression, or physical conditions associated with the disease affecting the patient.

As used herein the terms “administering” or “administration” refer to the act of injecting or otherwise physically delivering a substance as it exists outside the body (e.g., an inhibitor of IRE1α) into the subject, such as by mucosal, intradermal, intravenous, subcutaneous, intramuscular delivery and/or any other method of physical delivery described herein or known in the art. When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof.

Another aspect of the invention relates to a therapeutic composition comprising clozapine for use in the treatment of psychosis episode comprising the step of i) determining if the patient will respond to amisulpride or olanzapine according to the method of the invention and ii) administering the therapeutic composition when the patient is determined as a non-responder of amisulpride or olanzapine.

Any therapeutic agent of the invention may be combined with pharmaceutically acceptable excipients, and optionally sustained-release matrices, such as biodegradable polymers, to form therapeutic compositions. “Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The form of the pharmaceutical compositions, the route of administration, the dosage and the regimen naturally depend upon the condition to be treated, the severity of the illness, the age, weight, and sex of the patient, etc.

The pharmaceutical compositions of the invention can be formulated for a topical, oral, intranasal, parenteral, intraocular, intravenous, intramuscular, intrathecal or subcutaneous administration and the like.

Particularly, the pharmaceutical compositions contain vehicles which are pharmaceutically acceptable for a formulation capable of being injected (like lipiodol, gelfoam, ivalon). These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The doses used for the administration can be adapted as a function of various parameters, and in particular as a function of the mode of administration used, of the relevant pathology, or alternatively of the desired duration of treatment.

In addition, other pharmaceutically acceptable forms include, e.g. tablets or other solids for oral administration; time release capsules; and any other form currently can be used.

The invention will be further illustrated by the following figures and examples. However, these examples and figures should not be interpreted in any way as limiting the scope of the present invention.

### FIGURES

FIG. 1. Flow chart of the study. Through the OPTiMiSE study, 491 patients were included with a first episode of psychosis and 453 started amisulpride medication. The RNA-Seq analysis has been conducted on a subsample of 188 subjects. An independent sample of 24 subjects from the OPTiMiSE cohort has been used for the replication study. N, number of subjects; qRT-PCR, quantitative reverse transcription polymerase chain reaction.

FIG. 2. Receiver operator characteristic curves from logistic regression models predicting good or bad response to amisulpride treatment. A. The curve (1) represents the model combining the gene expression level of ALPL, CA4, DHRS13 and HOMER3. The curve (2) represents the model combining the age, the positive PANSS score (PPANSS) and the duration of untreated psychosis (DUP). The curve (1) represents the model combining the ALPL, CA4, DHRS13 and HOMER3 gene expression level as well as the age, the PPANSS and the DUP at inclusion. Area under the curve (AUC) are indicated for each model. B. The curve (1) represents the model combining the gene expression level of ALPL, CA4, DGAT2, DHRS13, HOMER3 and WLS. The curve (2) represents the model combining the age, the sex, the positive PANSS score and the DUP. The curve (3) represents the model combining the ALPL, CA4, DGAT2, DHRS13, HOMER3 and WLS gene expression level as well as the age, the sex, the Positive PANSS and the DUP at inclusion. AUC are indicated for each model.

TABLE 1
Genes differentially expressed after 4 weeks of treatment with amisulpride. Brain expression was defined when more than 1 transcript
per million was found in brain tissue of GTEx portal (https://www.gtexportal.org/). Significant p-value resisting to a Benjamini-Hochberg
false discovery rate (FDR) of 0.1 are shown in bold. FC, fold-change at week 4 relative to the expression level at inclusion.

| | | | | | | | | Spearman correlation | | | | | | | |
|---|---|---|---|---|---|---|---|---|---|---|---|---|---|---|---|
| | | | | | | | | between gene | | | | | | | |
| | | Good responders | | | Poor responders | | | expression changes | | | Protein | | Replication | | |
| | | (n = 113) | | | (n = 75) | | | and symptom changes | | | coding | Brain | sample (n = 24) | | |
| | Gene name | FC | p | FDR | FC | p | FDR | rho | p | FDR | genes | expression^{a} | FC | p | FDR |
| Down- | CA4 | 0.76 | 5.67E−09 | 0.0001 | 1.07 | 0.23 | 0.72 | −0.28 | 0.0001 | 0.0008 | yes | yes | 0.71 | 0.01 | 0.06 |
| regulated | AC073172.1 | 0.72 | 6.58E−07 | 0.005 | 0.97 | 0.68 | 0.92 | −0.18 | 0.01 | 0.03 | no | — | — | — | — |
| genes | NFE4 | 0.83 | 1.94E−06 | 0.01 | 0.95 | 0.25 | 0.73 | −0.21 | 0.005 | 0.02 | yes | no | — | — | — |
| | GALNT14 | 0.78 | 1.28E−05 | 0.05 | 1.04 | 0.48 | 0.85 | −0.22 | 0.002 | 0.01 | yes | yes | 0.76 | 0.02 | 0.05 |
| | AP000640.1 | 0.84 | 1.53E−05 | 0.05 | 1.00 | 0.96 | 0.99 | −0.19 | 0.008 | 0.02 | no | yes | — | — | — |
| | KAZN | 0.83 | 2.39E−05 | 0.05 | 0.94 | 0.22 | 0.71 | −0.15 | 0.05 | 0.06 | yes | yes | 0.67 | 0.01 | 0.05 |
| | KIAA0319 | 0.83 | 2.81E−05 | 0.05 | 0.94 | 0.17 | 0.67 | −0.17 | 0.02 | 0.03 | yes | yes | 0.77 | 0.08 | 0.12 |
| | NLRP12 | 0.87 | 5.46E−05 | 0.07 | 1.08 | 0.06 | 0.55 | −0.24 | 0.001 | 0.008 | yes | no | — | — | — |
| | FBXL13 | 0.85 | 5.93E−05 | 0.07 | 0.92 | 0.05 | 0.52 | −0.14 | 0.06 | 0.08 | yes | no | — | — | — |
| | ALPL | 0.79 | 7.52E−05 | 0.07 | 1.09 | 0.22 | 0.71 | −0.23 | 0.002 | 0.01 | yes | yes | 0.77 | 0.009 | 0.09 |
| | AL391832.3 | 0.88 | 8.76E−05 | 0.07 | 1.00 | 0.96 | 0.99 | −0.21 | 0.004 | 0.02 | no | — | — | — | — |
| | P4HA2 | 0.89 | 9.46E−05 | 0.07 | 1.04 | 0.38 | 0.80 | −0.20 | 0.007 | 0.02 | yes | yes | — | — | — |
| | DGAT2 | 0.87 | 9.65E−05 | 0.07 | 1.08 | 0.07 | 0.56 | −0.28 | 0.0001 | 0.002 | yes | yes | 0.73 | 0.08 | 0.11 |
| | AC092171.4 | 0.85 | 1.01E−04 | 0.07 | 1.06 | 0.31 | 0.77 | −0.20 | 0.007 | 0.02 | no | yes | — | — | — |
| | AC132872.1 | 0.81 | 1.01E−04 | 0.07 | 1.05 | 0.48 | 0.85 | −0.22 | 0.003 | 0.01 | no | yes | — | — | — |
| | LINC00963 | 0.91 | 1.04E−04 | 0.07 | 1.08 | 0.01 | 0.51 | −0.28 | 0.0001 | 0.001 | no | yes | — | — | — |
| | WLS | 0.87 | 1.10E−04 | 0.07 | 0.98 | 0.58 | 0.89 | −0.17 | 0.02 | 0.03 | yes | yes | 0.85 | 0.15 | 0.19 |
| | AL133351.4 | 0.88 | 1.12E−04 | 0.07 | 1.04 | 0.28 | 0.75 | −0.17 | 0.02 | 0.03 | no | — | — | — | — |
| | NLRP6 | 0.81 | 1.12E−04 | 0.07 | 1.09 | 0.19 | 0.68 | −0.14 | 0.05 | 0.07 | yes | no | — | — | — |
| | PLB1 | 0.85 | 1.12E−04 | 0.07 | 0.99 | 0.73 | 0.93 | −0.11 | 0.14 | 0.14 | yes | yes | — | — | — |
| | ALG1L13P | 0.89 | 1.33E−04 | 0.07 | 1.02 | 0.74 | 0.94 | −0.17 | 0.02 | 0.03 | no | yes | — | — | — |
| | HOMER3 | 0.84 | 1.48E−04 | 0.08 | 1.09 | 0.16 | 0.65 | −0.14 | 0.06 | 0.07 | yes | yes | 0.84 | 0.04 | 0.07 |
| | DHRS13 | 0.87 | 1.75E−04 | 0.09 | 1.04 | 0.30 | 0.76 | −0.18 | 0.01 | 0.03 | yes | yes | 0.82 | 0.02 | 0.04 |
| Up- | C15orf54 | 1.18 | 1.68E−05 | 0.05 | 1.07 | 0.15 | 0.64 | 0.10 | 0.19 | 0.21 | no | no | — | — | — |
| regulated | TRPC6 | 1.26 | 3.00E−05 | 0.05 | 0.97 | 0.63 | 0.91 | 0.18 | 0.01 | 0.03 | yes | yes | — | — | — |
| genes | SLC4A4 | 1.15 | 4.85E−05 | 0.07 | 0.93 | 0.13 | 0.62 | 0.18 | 0.02 | 0.03 | yes | yes | 0.84 | 0.31 | 0.34 |
| | P2RY12 | 1.14 | 5.79E−05 | 0.07 | 1.10 | 0.03 | 0.51 | 0.04 | 0.62 | 0.64 | yes | yes | — | — | — |
| | CYSLTR2 | 1.13 | 1.07E−04 | 0.07 | 1.01 | 0.88 | 0.98 | 0.11 | 0.13 | 0.14 | yes | yes | — | — | — |
| | CXCR6 | 1.14 | 1.08E−04 | 0.07 | 1.03 | 0.46 | 0.84 | 0.11 | 0.12 | 0.15 | yes | no | — | — | — |
| | FAT1 | 1.27 | 1.19E−04 | 0.07 | 1.20 | 0.01 | 0.51 | 0.06 | 0.38 | 0.41 | yes | yes | — | — | — |
| | ACSL5 | 1.06 | 1.33E−04 | 0.08 | 0.99 | 0.52 | 0.86 | 0.18 | 0.01 | 0.03 | yes | yes | 1.06 | 0.67 | 0.67 |
| | GUCY1B3 | 1.14 | 1.44E−04 | 0.08 | 1.05 | 0.25 | 0.73 | 0.12 | 0.09 | 0.10 | yes | yes | — | — | — |

TABLE 2
Gene differential expression at inclusion between future good
and poor responders to amisulpride treatment. Significant p-
values resisting to a Benjamini-Hochberg false discovery rate
(FDR) of 0.1 are shown in bold. FC, expression fold-change.

| | Difference between future good and poor | | |
|---|---|---|---|
| | responders at inclusion | | |
| Gene name | FC | p-value | FDR |
| ALPL | 1.32 | 0.003 | 0.02 |
| DHRS13 | 1.18 | 0.003 | 0.02 |
| HOMER3 | 1.18 | 0.01 | 0.04 |
| DGAT2 | 1.15 | 0.02 | 0.04 |
| WLS | 1.16 | 0.05 | 0.08 |
| CA4 | 1.16 | 0.05 | 0.08 |
| GALNT14 | 1.16 | 0.13 | 0.19 |
| ACSL5 | 0.99 | 0.62 | 0.78 |
| KIAA0319 | 1.02 | 0.75 | 0.84 |
| KAZN | 1.00 | 0.97 | 0.97 |

Significant p-values resisting to a Benjamini-Hochberg false discovery rate (FDR) of 0.1 are shown in bold. FC, expression fold-change.

TABLE 3
Predictive value of models based on gene expression and clinical data at inclusion.

| | AUC | | | | | | |
|---|---|---|---|---|---|---|---|
| response~ | [95% CI] | p^{a} | Sensitivity | Specificity | PPV | NPV | Accuracy |
| ALPL | 0.589 | 0.04 | 0.68 | 0.56 | 0.70 | 0.54 | 0.63 |
| | [0.505; 0.673] | | | | | | |
| DHRS13 | 0.613 | 0.008 | 0.46 | 0.75 | 0.73 | 0.48 | 0.57 |
| | [0.533; 0.693] | | | | | | |
| HOMER3 | 0.604 | 0.01 | 0.65 | 0.56 | 0.69 | 0.51 | 0.61 |
| | [0.524; 0.685] | | | | | | |
| DGAT2 | 0.634 | 0.002 | 0.37 | 0.85 | 0.79 | 0.47 | 0.56 |
| | [0.555; 0.713] | | | | | | |
| WLS | 0.608 | 0.01 | 0.56 | 0.67 | 0.72 | 0.50 | 0.60 |
| | [0.527; 0.688] | | | | | | |
| CA4 | 0.622 | 0.005 | 0.50 | 0.72 | 0.73 | 0.49 | 0.59 |
| | [0.543; 0.702] | | | | | | |
| ALPL*DHRS13*HOMER3 | 0.791 | 0.77 | 0.77 | 0.68 | 0.78 | 0.66 | 0.73 |
| *DGAT2*WLS*CA4 | [0.728; 0.853] | | | | | | |
| Age + Sex + DUP + PPANSS | 0.716 | 0.0001 | 0.74 | 0.67 | 0.77 | 0.63 | 0.71 |
| | [0.640; 0.791] | | | | | | |
| ALPL + Age + Sex + DUP + PPANSS | 0.751 | <10⁻⁴ | 0.73 | 0.73 | 0.80 | 0.64 | 0.73 |
| | [0.678; 0.824] | | | | | | |
| DHRS13 + Age + Sex + DUP + PPANSS | 0.740 | <10⁻⁴ | 0.88 | 0.51 | 0.73 | 0.75 | 0.73 |
| | [0.666; 0.813] | | | | | | |
| HOMER3 + Age + Sex + DUP + PPANSS | 0.722 | <10⁻⁴ | 0.71 | 0.73 | 0.80 | 0.62 | 0.72 |
| | [0.647; 0.797] | | | | | | |
| DGAT2 + Age + Sex + DUP + PPANSS | 0.743 | <10⁻⁴ | 0.65 | 0.77 | 0.81 | 0.60 | 0.70 |
| | [0.670; 0.816] | | | | | | |
| WLS + Age + Sex + DUP + PPANSS | 0.738 | <10⁻⁴ | 0.82 | 0.63 | 0.77 | 0.70 | 0.74 |
| | [0.664; 0.812] | | | | | | |
| CA4 + Age + Sex + DUP + PPANSS | 0.741 | <10⁻⁴ | 0.59 | 0.81 | 0.83 | 0.57 | 0.68 |
| | [0.669; 0.812] | | | | | | |
| ALPL*DHRS13*HOMER3 | 0.938 | 0.02 | 0.88 | 0.89 | 0.92 | 0.84 | 0.89 |
| *DGAT2*WLS*CA4 + | [0.905; 0.971] | | | | | | |
| Age + Sex + DUP + PPANSS | | | | | | | |

^{a}p-values have been estimated after 10,000 permutations of the response status in 188 individuals. AUC, area under the curve; CI, confidence interval; DUP, duration of untreated psychosis; NPV, negative predictive value; PPANSS, positive PANSS score; PPV, positive predictive value.

TABLE 4
Characteristics of patients. Quantitative variables are expressed either with mean (standard
deviation) or with median (inter quartile range). SZ, schizophrenia; SD, schizophreniform
disorder; SA, schizoaffective disorder; DUP, duration of untreated psychosis.

| | Good responders | | Poor responders | | | |
|---|---|---|---|---|---|---|
| At inclusion | (n = 113) | | (n = 75) | | Statistics | p-value |
| Age (years) | 25.2 | (9.7) | 23.1 | (7.4) | U = 5134.5 | 0.01 |
| Female (%) | 36.3 | | 28 | | χ² =1.40, df = 1 | 0.24 |
| Current smokers (%) | 48.7 | | 42.7 | | χ² = 0.65, df = 1 | 0.42 |
| Recreational drugs (%) | 45.1 | | 45.3 | | χ² = 0.00, df = 1 | 0.98 |
| Diagnosis: SZ/SD/SA (%) | 42.5/50.4/7.1 | | 58.7/34.7/6.7 | | χ² = 4.97, df = 2 | 0.08 |
| DUP (months) | 3 | (4) | 6 | (10) | U = 3132.5 | 0.03 |
| PANSS-Total | 80.7 | (17.9) | 75.7 | (20) | t = 1.75, df = 186 | 0.07 |
| PANSS-Positive | 21.1 | (5.4) | 18.6 | (5.9) | t = 3.00, df = 186 | 0.003 |
| PANSS-Negative | 19.8 | (6.4) | 19.9 | (7.6) | t = −0.07, df = 186 | 0.95 |
| PANSS-General | 39.8 | (9.1) | 37.2 | (9.8) | t = 1.83, df = 186 | 0.07 |

### MATERIEL AND METHODS

### Subject

The cohort analysed in this study was obtained from the OPTiMiSE clinical trial (http://www.optimisetrial.eu) (29). After providing written informed consent, 491 patients with a first episode psychosis were included and 453 started medication. All patients were diagnosed with schizophrenia, schizophreniform disorder or schizoaffective disorder according to the Diagnostic and Statistical Manual of Mental Disorders, Fourth Edition (DSM-IV) and confirmed on the basis of the Mini International Neuropsychiatric Interview Plus (M.I.N.I. Plus) (30) (table 4). At baseline, patients were medication-naïve or minimally-treated (any antipsychotic medication used for more than 2 weeks in the previous year or for a total of 6 weeks in their lifetime) and were ill for no longer than 2 years. Only subjects involved in the single-treatment arm (phase 1) of the trial were considered for this study (29). All patients were treated for 4 weeks with amisulpride (200-800 mg/day orally). At the end of the 4-week treatment, the blood concentration of antipsychotic used has been determined. This measurement provided a reliable indication of the treatment compliance of individual patients.

### RNA Extraction

Total RNA was extracted from blood samples, collected in PAXgene Blood RNA tubes, with the PAXgene Blood miRNA Kit (QIAGEN GmbH, Hilden, Germany) using a standard protocol on QlAcube robot (QIAGEN). A DNase digestion procedure was added after extraction. Total RNA was purified using the RNA Clean & Concentrator™-5 kit following the manufacturer's instructions (Zymo Research Corp., Irvine, USA). RNA quantification was performed on a NanoDrop™ 8000 spectrophotometer, in duplicate. The quality and integrity of the RNA samples were assessed using an Agilent Bioanalyzer 2100 and the RNA6000 Nano Labchip kit (Agilent Technologies). Only samples with an RNA integrity number (RIN) higher than 7 were selected. Three hundred and seventy-six RNA samples fulfilled quality control criteria for RNA-seq analyses, corresponding to two RNA samples (one at inclusion and one after 4 weeks of treatment) for 188 subjects. Forty-eight additional RNAs were available for replication studies, in 24 independent subjects.

### RNA Sequencing

All libraries were prepared using the Illumina TruSeq Stranded Total RNA with Ribo-Zero Globin kit (Illumina Inc., San Diego, Calif., U.S.A.), which removes ribosomal RNA and globin mRNA. Libraries were prepared using one microgram of total RNA as initial input and following the standard protocols. After library quality control, 2×100 bp paired-end (PE) sequencing was performed on a HiSeq4000 system (Illumina), pooling a maximum of 6 samples on each lane, in order to reach 40 to 50 million of PE-reads for each sample. The Illumina pipeline was used to generate raw RNA sequencing data (fastq files) for each sample. Sequence quality controls were performed, using FastQC and in-house bioinformatics pipelines, from a sampling of 2×10 million reads in order to assess the levels of read duplicates, adapters, remaining rRNA and the GC content. Reads were trimmed for adapters and low-quality bases (Phred quality score <30) using Trimmomatic software (v.0.32). Reads were then mapped to the Genome Reference Consortium human genome assembly 37 (GRCh37) reference genome (hg19) using Tophat software (v.2.0.13). Read mapping quality was assessed using RNA-SeQC software. Then, gene-level quantification in read counts was performed by HTSeq software (v.0.6.1), using gene annotation from Ensembl v.86. All downstream analyses were conducted using the statistical software R (v.3.2.4) and Bioconductor R packages. Genes with counts-per million (cpm) below 1 in more than two third of the samples were considered unexpressed and removed from the analysis using edgeR package (v.3.12.1). Raw gene counts matrix has been normalized to account for differences in sequencing depth and RNA composition using the “median ratio” method implemented in the DESeq2 package (v.1.16.1). To avoid batch effects as confounding factors, all subjects had their 2 time-points in a same batch. Considering the whole normalized set of expressed genes, principal component analysis (PCA) has been performed using the FactoMineR package (v.1.39) to evaluate the efficacy of DESeq2 normalization and for identifying outliers.

### Validation of Expression Data by Quantitative Reverse Transcription Polymerase Chain Reaction (qRT-PCR)

Five-hundred nanograms of total RNA was reverse-transcribed using the High-Capacity cDNA Reverse Transcription kit following standard protocol (Thermo Fisher Scientific, Waltham, Mass., U.S.A.). Mixtures of the cDNAs and the TaqMan Universal PCR Master Mix were further loaded in the 384-well low density TaqMan array microfluidic cards (Thermo Fisher Scientific). Real-time PCR reactions were then carried out in an ABI Prism 7900HT sequence detection system (Thermo Fisher Scientific) using manufacturer's instructions. Each assay was carried out in duplicate and threshold cycle (Ct) values were automatically calculated by the SDS 2.2 software (Thermo Fisher Scientific), after having manually set the analysis threshold. Two reference genes (18S, and GUSB) with various expression levels were included in the analyses to perform a relative RNA quantification, using the most suitable reference gene.

### Statistical Analyses

All statistical analyses were conducted with R (v.3.2.4). All variables normally distributed were expressed as mean±standard deviation (SD) and comparisons between conditions were examined by t-tests and one-way analyses of variance (ANOVAs). Not normally distributed variables were expressed as median and interquartile range (IQR) and between-group comparisons were analysed by Mann-Whitney and Kruskal-Wallis tests. Differential expression before and after treatment was performed by a paired-analysis with the DESeq function and the Wald test using two explanatory factors in the experimental design, participants and time (two time-points per subject). For gene expression levels before treatment, we corrected putative bias from library preparation batches as well as from the inclusion site. We then compared good and poor responders using the same function and a statistical model adjusted for age, sex, ancestry, DUP, positive PANSS score at inclusion and centre in which individuals had been recruited, these features either having been shown to be significantly different between future good and poor responders (Table 4) or being putative confounding factors. Significantly DEGs between the two-time points were defined as having a Benjamini-Hochberg (BH) corrected p-value lower than 0.1.

For the qRT-PCR analysis, ΔCt values of each gene of interest were calculated before and after treatment and then normalized by subtracting the ΔCt values of the reference gene to generate ΔΔCt values. To select genes with significant expression changes after treatment, the medians of fold changes (2-ΔΔCt) were subsequently compared to a reference (Me=1) using Wilcoxon Rank Sum Test, and an adjusted p-values below 0.1 was considered to be significant.

Receiver operating characteristic (ROC) curves were drawn using the pROC package (v.1.14.0) to assess the performance of the treatment response prediction based on the expression level of genes differentially expressed at inclusion between subjects who will have a good response and those who will have a poor response to amisulpride treatment. We thus performed 10,000 permutations of the outcome labels and calculated the probability of getting our observed area under the curve (AUC) by chance.

The statistical power was estimated using the PROPER package (v.1.14.1) (Wu et al., Bioinformatics, 2014, doi: 10.1093/bioinformatics/btu640.) with 1000 simulations based on lymphoblastoid cell lines expression data from CEU individuals of the HapMap project. We considered 16,204 genes that were expressed and 10% genes that were differentially expressed. Only genes with more than 10 counts were included. The statistical power was estimated using 0.1 as an FDR threshold.

### RESULTS

### Genes are Differentially Expressed After Amisulpride Treatment in Good Responders Only

A clinical response to treatment can be defined according to many criteria. The distribution of the total PANSS score change over 4 weeks of treatment revealed an admixture of two subpopulations with a Gaussian distribution (data not shown). In this model, 56% were good responders, with a mean reduction of total PANSS score of 36.3% (SD=12.1), and 44% were poor responders, with a mean PANSS total reduction of 13.0% (SD=18.5). When the intersection of the two subpopulations was used to distinguish good and poor responders, 113 patients had a more than 20% reduction in total PANSS score and 75 patients had a less than 20% reduction, which was consistent with previous studies (36-39). Note that good responders were slightly older than poor responders with a lower duration of untreated psychosis and more positive symptoms (data not shown).

In order to identify molecular mechanisms associated with treatment outcome, we compared gene expression levels at baseline and after 4 weeks of treatment with amisulpride. We thus conducted an RNA-seq analysis before and after treatment on total RNA from PBMCs of 188 subjects. We generated an average of 47 million of 100bp paired-end reads per sample. After normalization and quality control, 16,204 genes were expressed in PBMCs before and after treatment. Visual inspection of the first two dimensions of the PCA plot performed on the whole set of genes revealed a homogeneous cluster (data not shown). Comparison of gene expression levels before and after treatment revealed that good responders only showed 32 DEGs that survived to BH-correction for multiple testing (FDR threshold of 0.1). Nine of these genes were up-regulated and 23 were down-regulated (Table 1). Although the population of poor responders was smaller than the one of good responders, we estimated that we had a statistical power of 92% to detect a fold change of 1.06, which was the minimum difference that we observed in the 32 genes differentially expressed after treatments in good responders. This demonstrates that the difference observed between good and poor responders resulted from the response status.

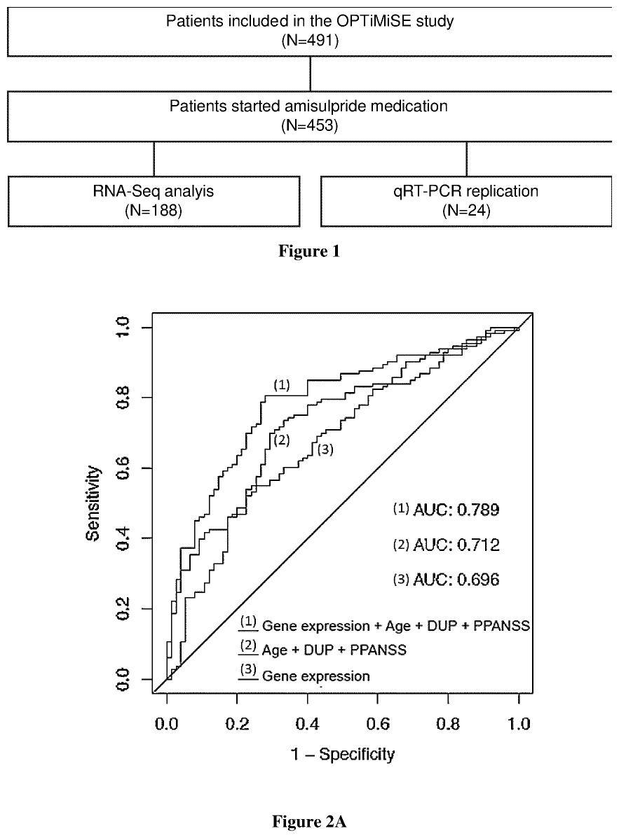

To validate these DEGs, we used qRT-PCR in 24 independent subjects from the OPTiMiSE cohort, who were not included in the RNA-seq analyses and with a more than 20% improvement in total PANSS score after treatment (FIG. 1). We selected only brain-expressed protein-coding genes that were differentially expressed after treatment in good responders only. In addition, we restricted our selection to genes for which the expression change was correlated with symptom improvement (Table 1). Although they did not show an FDR lower than 0.1 in bad responders, three genes (FAT1, FBXL13 and P2RY12) were thus excluded because they had a fold change close to those of good responders and a nominal p-value lower than 0.05. In addition, two genes (P4HA2 and TRPC6) were removed from replication studies, because they were not detectable by qRT-PCR. Eleven genes were thus tested in the replication sample. Despite a small sample size limiting our statistical power (80% of probability of detecting a fold change higher than 1.13 or lower than 0.88), 6 genes (ALPL, CA4, DHRS13, GALNT14, KAZN and HOMER3) showed a significant difference in expression levels before and after treatment that resisted to BH-correction for multiple testing (FDR<0.1) (Table 1).

### Gene Expression Level at Baseline Predicts Treatment Response

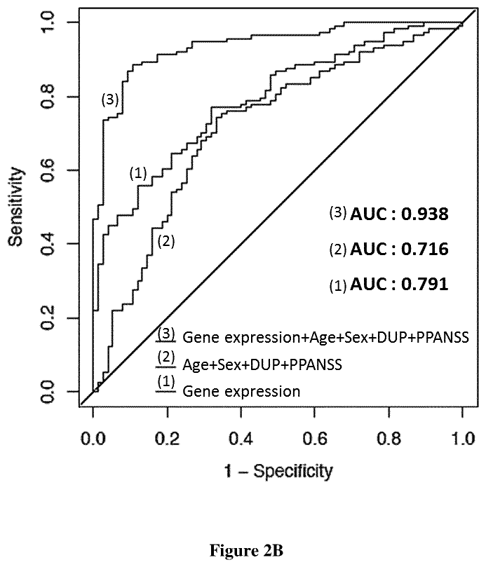

To test whether the 6 genes (ALPL, CA4, DHRS13, GALNT14, KAZN and HOMER3) that were differentially expressed in the replication sample could be used as biomarkers of treatment response, we compared their expression levels before treatment between patients who will show a good response to treatment after 4 weeks (n=113) and those who will not (n=75). In addition to the 6 genes (ALPL, CA4, DHRS13, GALNT14, HOMER3 and KAZN) that were differentially expressed in the replication sample, we included 4 genes (ACSL5, DGAT2, KIAA0319 and WLS) for which the expression fold-changes were similar between the RNA-Seq and the qRTPCR analyses (Table 1). Six out of the 10 genes (ALPL, CA4, DHRS13, DGAT2, HOMER3 and WLS)) were significantly overexpressed in patients who will respond to treatment (Table 2). We then estimated the expression level-based predictive performance for these 6 genes. Area under the curve (AUC) varied between 0.589 and 0.634 according to the gene (Table 3). Ten-thousand label permutations among the 188 individuals showed this prediction was significantly higher than random prediction for the 6 genes. As we observed clinical differences between future good and poor responders, we adjusted our model in a multivariate analysis including the age at inclusion, the sex, the duration of untreated psychosis (months) and the positive PANSS score at inclusion. Addition of clinical criteria improved our models for the 6 genes (Table 3). Note, the predictive values combining gene expression and clinical data for each gene was higher than those calculated for gene expression alone or clinical data alone. Finally, we computed a multivariate model considering the expression level of the 6 genes with clinical data and estimated that our model was able to discriminate between good and poor responders with 93.8% chance. As observed on FIG. 2A and 2B, the combination of both gene expression level (of 4 genes and 6 genes, respectively) at inclusion and clinical features at inclusion considerably improve the overall performance of models based only on gene expression (Z=2.63, p=0.009 and Z=4.82, =7×10⁻⁷ respectively) or on clinical data alone (Z=3.15, p=0.002 and Z=6.14, p=4×10⁻¹⁰ respectively), showing the importance of combining clinical and biological data to predict treatment response in first episode psychoses.

### Conclusion

Our first hypothesis was that there would be differences in gene expression between good and bad responders to amisulpride. In this study, we found that only patients with a more than 20% improvement in total PANSS score after 4 weeks of amisulpride treatment showed a differential gene expression after treatment. These results show both that antipsychotic medication can affect gene expression in peripheral mononuclear cells, as previously reported (25-27, 40, 41), and that this expression varies according to the effectiveness of treatment. The majority (23 out of 32) of our significant DEGs were down regulated after treatment. This is consistent with data from previous studies using other antipsychotic medications, in which patients with schizophrenia exhibited a greater dysregulation of gene expression than after treatment when compared with unaffected subjects (25, 26). This suggests that antipsychotic medication may normalize the altered expression of genes implicated in schizophrenia.

Our second hypothesis was to determine whether genes for which the expression level changes after treatment in good responders might be used to predict treatment outcome. Six out of the 10 DEGs that we tested showed an overexpression at inclusion in patients whose symptomatology improved after 4-week treatment. This is consistent with data from previous studies using other antipsychotic medications, reporting that genes overexpressed in subjects with schizophrenia when compared with controls, were downregulated after antipsychotic medication [25,26]. As expected, the six genes that we identified in our cohort were downregulated after treatment, suggesting that antipsychotic medication may normalize the altered expression of genes implicated in schizophrenia.

The 4 genes for which we confirmed the differential expression by replication on an independent sample had previously been reported as being differentially expressed in brain of patients with schizophrenia [26,43]. In particular, ALPL has been previously reported to be overexpressed in amygdala of individuals with schizophrenia as well as in blood of drug-naïve patients [43]. Consistently with what we observed in our cohort, its expression has been shown to decrease after treatment with different atypical antipsychotics [25], suggesting its expression level might be used for treatment response prediction irrespective of the drugs took by affected individuals. Moreover, we observed similar predictive values for the four replicated genes (ALPL, DHRS13, HOMER3 and CA4) as well as for 2 additional genes (DGAT2 and WLS) that were differentially expressed at inclusion between the future good and poor responders. For all of them, we were able to increase the accuracy of our models combining clinical data and gene expression at inclusion. Although the proteins encoded by these genes were not known to interact together or to be involved in a single functional pathway, the best model was observed when combining the expression level of the six genes and was improved again when we added clinical features. These results are consistent with the polygenic hypothesis of schizophrenia and demonstrate the importance of combining biological and clinical markers to develop precision medicine in psychiatry.

We have recently shown that change from amisulpride to olanzapine did not improve outcome for most of the patients [28]. These biomarkers may thus help in selecting patients to treat earlier with clozapine. Further replications on independent cohorts are now needed to confirm our results and determine if our biomarker-based treatment response prediction might be relevant for other antipsychotic medications.

Altogether, our results identified new mechanisms to explain symptom improvement after amisulpride medication and propose new blood biomarkers that, in combination with selected clinical symptoms, may help in predicting treatment outcome in first episode psychoses.

### REFERENCES

Throughout this application, various references describe the state of the art to which this invention pertains. The disclosures of these references are hereby incorporated by reference into the present disclosure.

1. Kahn R S, Fleischhacker W W, Boter H, Davidson M, Vergouwe Y, Keet I P M, et al. (2008): Effectiveness of antipsychotic drugs in first-episode schizophrenia and schizophreniform disorder: an open randomised clinical trial. Lancet. 371:1085-1097.

2. Leucht S, Cipriani A, Spineli L, Mavridis D, Örey D, Richter F, et al. (2013): Comparative efficacy and tolerability of 15 antipsychotic drugs in schizophrenia: a multiple-treatments meta-analysis. The Lancet. 382:951-962.

3. Lieberman J A, Stroup T S, McEvoy J P, Swartz M S, Rosenheck R A, Perkins D O, et al. (2005): Effectiveness of antipsychotic drugs in patients with chronic schizophrenia. N Engl J Med. 353:1209-1223.

4. Case M, Stauffer V L, Ascher-Svanum H, Conley R, Kapur S, Kane J M, et al. (2011): The heterogeneity of antipsychotic response in the treatment of schizophrenia. Psychol Med. 41:1291-1300.

5. Fond G, Godin O, Boyer L, Berna F, Andrianarisoa M, Coulon N, et al. (2018): Chronic low-grade peripheral inflammation is associated with ultra resistant schizophrenia. Results from the FACE-SZ cohort. European archives of psychiatry and clinical neuroscience.

6. Arranz M J, Munro J C (2011): Toward understanding genetic risk for differential antipsychotic response in individuals with schizophrenia. Expert Rev Clin Pharmacol. 4:389-405.

7. Howes O D, Vergunst F, Gee S, McGuire P, Kapur S, Taylor D (2012): Adherence to treatment guidelines in clinical practice: study of antipsychotic treatment prior to clozapine initiation. Br J Psychiatry. 201:481-485.

8. Arranz M J, de Leon J (2007): Pharmacogenetics and pharmacogenomics of schizophrenia: a review of last decade of research. Molecular Psychiatry. 12:707-747.

9. Arranz M, Collier D, Sodhi M, Ball D, Roberts G, Price J, et al. (1995): Association between clozapine response and allelic variation in 5-HT2A receptor gene. Lancet. 346:281-282.

10. Brandl E J, Kennedy J L, Müller D J (2014): Pharmacogenetics of antipsychotics. Can J Psychiatry. 59:76-88.

11. Need A C, Keefe R S E, Ge D, Grossman I, Dickson S, McEvoy J P, et al. (2009): Pharmacogenetics of antipsychotic response in the CATIE trial: a candidate gene analysis. Eur J Hurn Genet. 17:946-957.

12. Schäfer M, Rujescu D, Giegling I, Guntermann A, Erfurth A, Bondy B, et al. (2001): Association of Short-Term Response to Haloperidol Treatment With a Polymorphism in the Dopamine D2 Receptor Gene. AJP. 158:802-804.

13. Vehof J, Burger H, Wilffert B, Al Hadithy A, Alizadeh B Z, Snieder H, et al. (2012): Clinical response to antipsychotic drug treatment: association study of polymorphisms in six candidate genes. Eur Neuropsychopharmacol. 22:625-631.

14. Zhang J-P, Lencz T, Malhotra A K (2010): Dopamine D2 receptor genetic variation and clinical response to antipsychotic drug treatment: A meta-analysis. Am J Psychiatry. 167:763-772.

15. Le Clerc S, Taing L, Fond G, Meary A, Llorca P M, Blanc O, et al. (2015): A double amino-acid change in the HLA-A peptide-binding groove is associated with response to psychotropic treatment in patients with schizophrenia. Transl Psychiatry. 5:e608.

16. Clark S L, Souza R P, Adkins D E, Aberg K, Bukszár J, McClay J L, et al. (2013): Genome-wide association study of patient-rated and clinician-rated global impression of severity during antipsychotic treatment. Pharmacogenet Genomics. 23:69-77.

17. Lavedan C, Licamele L, Volpi S, Hamilton J, Heaton C, Mack K, et al. (2008): Association of the NPAS3 gene and five other loci with response to the antipsychotic iloperidone identified in a whole genome association study. Molecular Psychiatry. 14:804-819.

18. Li J, Yoshikawa A, Brennan M D, Ramsey T L, Meltzer H Y (2017): Genetic predictors of antipsychotic response to lurasidone identified in a genome wide association study and by schizophrenia risk genes. Schizophr Res.

19. Li Q, Wineinger N E, Fu D-J, Libiger O, Alphs L, Savitz A, et al. (2017): Genome-wide association study of paliperidone efficacy. Pharmacogenet Genomics. 27:7-18.

20. McClay J L, Adkins D E, Aberg K, Bukszár J, Khachane A N, Keefe RSE, et al. (2011): Genome-wide pharmacogenomic study of neurocognition as an indicator of antipsychotic treatment response in schizophrenia. Neuropsychopharmacology. 36:616-626.

21. McClay J L, Adkins D E, Aberg K, Stroup S, Perkins D O, Vladimirov V I, et al. (2011): Genome-wide pharmacogenomic analysis of response to treatment with antipsychotics. Molecular Psychiatry. 16:76-85.

22. Sacchetti E, Magri C, Minelli A, Valsecchi P, Traversa M, Calza S, et al. (2017): The GRM7 gene, early response to risperidone, and schizophrenia: a genome-wide association study and a confirmatory pharmacogenetic analysis. Pharmacogenomics J. 17:146-154.

23. Stevenson J M, Reilly J L, Harris M S H, Patel S R, Weiden P J, Prasad K M, et al. (2016): Antipsychotic pharmacogenomics in first episode psychosis: a role for glutamate genes. Translational Psychiatry. 6:e739.

24. Yu H, Yan H, Wang L, Li J, Tan L, Deng W, et al. (2018): Five novel loci associated with antipsychotic treatment response in patients with schizophrenia: a genome-wide association study. Lancet Psychiatry. 5:327-338.

25. Crespo-Facorro B, Prieto C, Sainz J (2014): Schizophrenia gene expression profile reverted to normal levels by antipsychotics. Int J Neuropsychopharmacol. 18.

26. Kumarasinghe N, Beveridge N J, Gardiner E, Scott R J, Yasawardene S, Perera A, et al. (2013): Gene expression profiling in treatment-naive schizophrenia patients identifies abnormalities in biological pathways involving AKT1 that are corrected by antipsychotic medication. Int J Neuropsychopharmacol. 16:1483-1503.

27. Sainz J, Prieto C, Ruso-Julve F, Crespo-Facorro B (2018): Blood Gene Expression Profile Predicts Response to Antipsychotics. Front Mol Neurosci. 11.

28. Kahn R S, Winter van Rossum I, Leucht S, McGuire P, Lewis S W, Leboyer M, et al. (2018): Amisulpride and olanzapine followed by open-label treatment with clozapine in first-episode schizophrenia and schizophreniform disorder (OPTiMiSE): a three-phase switching study. Lancet Psychiatry.

29. Leucht S, Winter-van Rossum I, Heres S, Arango C, Fleischhacker W W, Glenthøj B, et al. (2015): The optimization of treatment and management of schizophrenia in Europe (OPTiMiSE) trial: rationale for its methodology and a review of the effectiveness of switching antipsychotics. Schizophr Bull. 41:549-558.

30. Sheehan D V, Lecrubier Y, Sheehan K H, Amorim P, Janays J, Weiller E, et al. (1998): The Mini-International Neuropsychiatric Interview (M.I.N.I.): the development and validation of a structured diagnostic psychiatric interview for DSM-IV and ICD-10. J Clin Psychiatry. 59 Suppl 20:22-33;quiz 34-57.

31. Delaneau O, Zagury J F, Marchini J (2013): Improved whole-chromosome phasing for disease and population genetic studies. Nat Methods. 10:5-6.

32. Durbin R (2014): Efficient haplotype matching and storage using the positional Burrows-Wheeler transform (PBWT). Bioinformatics. 30:1266-1272.

33. Huang J, Howie B, McCarthy S, Memari Y, Walter K, Min J L, et al. (2015): Improved imputation of low-frequency and rare variants using the UK10K haplotype reference panel. Nat Commun. 6:8111.

34. Genomes Project C, Auton A, Brooks L D, Durbin R M, Garrison E P, Kang H M, et al. (2015): A global reference for human genetic variation. Nature. 526:68-74.

35. Ascher-Svanum H, Nyhuis A W, Faries D E, Kinon B J, Baker R W, Shekhar A (2008): Clinical, functional, and economic ramifications of early nonresponse to antipsychotics in the naturalistic treatment of schizophrenia. Schizophr Bull. 34:1163-1171.

36. Emsley R, Rabinowitz J, Medori R (2006): Time course for antipsychotic treatment response in first-episode schizophrenia. Am J Psychiatry. 163:743-745.

37. Kinon B J, Chen L, Ascher-Svanum H, Stauffer V L, Kollack-Walker S, Sniadecki J L, et al. (2008): Predicting response to atypical antipsychotics based on early response in the treatment of schizophrenia. Schizophr Res. 102:230-240.

38. Lin C H, Lin H S, Lin S C, Kuo C C, Wang F C, Huang Y H (2018): Early improvement in PANSS-30, PANSS-8, and PANSS-6 scores predicts ultimate response and remission during acute treatment of schizophrenia. Acta Psychiatr Scand. 137:98-108.

39. Watanabe K, Taskesen E, Bochoven Av, Posthuma D (2017): Functional mapping and annotation of genetic associations with FUMA. Nature Communications. 8:1826.

40. Harrison R N S, Murray R M, Lee S H, Paya Cano J, Dempster D, Curtis C J, et al. (2016): Gene-expression analysis of clozapine treatment in whole blood of patients with psychosis. Psychiatric Genetics. 26:211-217.

41. Ota V K, Noto C, Gadelha A, Santoro M L, Silva P N, Melaragno M I, et al. (2013): Neurotransmitter receptor and regulatory gene expression in peripheral blood of Brazilian drug-naïve first-episode psychosis patients before and after antipsychotic treatment. Psychiatry Res. 210:1290-1292.

42. Choi S. W et al (2018): A guide to performing Polygenic Risk Score analyses. Biorxiv.

43. Sainz J, Mata I, Barrera J, Perez-Iglesias R, Varela I, Arranz M J, et al. Inflammatory and immune response genes have significantly altered expression in schizophrenia. Molecular Psychiatry. 2013;18(10):1056-57.

1. A method for predicting an antipsychotic treatment response of a patient in need thereof, comprising: i) determining, in a sample obtained from the patient, the expression level of at least one gene selected from the group consisting of AC073172.1, AC092171.4, AC132872.1, ACSL5, AL133351.4, AL391832.3, ALG1L13P, ALPL, AP000640.1, C15orf54, CA4, CXCR6, CYSLTR2, DGAT2, DHRS13, FAT1, FBXL13, GALNT14, GUCY1B3, HOMER3, KAZN, KIAA0319, LINC00963, NFE4, NLRP12, NLRP6, P2RY12, P4HA2, PLB1, SLC4A4, TRPC6, and WLS; ii) comparing the expression level of the at least one gene determined at step i) with a reference value and iii) concluding that the patient will not respond to antipsychotic treatment when the expression level determined at step i) is significantly different from the reference value.

2. The method according to claim 1, wherein the expression level of the genes ALPL, CA4, DHRS13 and HOMER3 are determined in step i) .

3. The method according to claim 1, wherein the expression level of the genes ALPL, CA4, DGAT2, DHRS13, HOMER3 and WLS are determined in step i).

4. The method according to claim 1, wherein the antipsychotic treatment is an amisulpride or olanzapine treatment.

5. The method according to claim 1, wherein the patient is a patient diagnosed with schizophrenia, schizophreniform disorder or schizoaffective disorder.

6. A method for predicting a response to an antipsychotic treatment of a patient suffering from a psychosis episode, comprising the steps of: i) determining, in a sample obtained from the patient, the expression level of at least one gene selected from the group consisting of AC073172.1, AC092171.4, AC132872.1, ACSL5, AL133351.4, AL391832.3, ALG1L13P, ALPL, AP000640.1, C15orf54, CA4, CXCR6, CYSLTR2, DGAT2, DHRS13, FAT1, FBXL13, GALNT14, GUCY1B3, HOMER3, KAZN, KIAA0319, LINC00963, NFE4, NLRP12, NLRP6, P2RY12, P4HA2, PLB1, SLC4A4, TRPC6, and WLS; ii) comparing the expression level of the at least one gene determined at step i) with a reference value, iii) regarding clinical data of said patient, wherein the clinical data are the age, the duration of untreated psychosis (DUP), the sex and/or the positive PANSS score of said patient, iv) calculating a clinical data score of the patient using the clinical data from step iii) according to a logistic regression defined from a learning antipsychotic responders cohort and v) concluding that the patient will not respond to the antipsychotic treatment when the expression level determined at step i) is significantly different from the reference value and when the clinical data score is lower than an optimal threshold.

7. The method according to claim 6, wherein the expression levels of the genes ALPL, CA4, DHRS13 and HOMER3 are determined in step i).

8. The method according to claim 6, wherein the expression levels of the genes ALPL, CA4, DHRS13, DGAT2, WLS and HOMER3 are determined in step i).

9. The method according to claim 6, wherein the antipsychotic treatment is an amisulpride or olanzapine treatment.

10. A method of treating a psychosis episode in patient in need thereof comprising i) determining if the patient will respond to amisulpride or olanzapine by
i) determining, in a sample obtained from the patient, the expression level of at least one gene selected from the group consisting of AC073172.1, AC092171.4, AC132872.1, ACSL5, AL133351.4, AL391832.3, ALG1L13P, ALPL, AP000640.1, C15orf54, CA4, CXCR6, CYSLTR2, DGAT2, DHRS13, FAT1, FBXL13, GALNT14, GUCY1B3, HOMER3, KAZN, KIAA0319, LINC00963, NFE4, NLRP12, NLRP6, P2RY12, P4HA2, PLB1, SLC4A4, TRPC6, and WLS;
ii) comparing the expression level of the at least one gene determined at step i) with a reference value, and, when the patient is a non-responder to amisulpride or olanzapine because the expression level determined at step i) is significantly different from the reference value,
iii) administering a therapeutically effective amount of clozapine to the patient.
